(19) United States
(12) Patent Application Publication
(10) Pub. No.: US 2024/0002805 A1
(43) Pub. Date: Jan. 4, 2024

(54) OPTOGENETIC MODULATION OF ACTION-POTENTIAL PROPERTIES

(71) Applicants: TECHNION RESEARCH & DEVELOPMENT FOUNDATION LIMITED, Haifa (IL); RAMBAN MEDTECH LTD., Haifa (IL)

(72) Inventors: Lior GEPSTEIN, Hifa (IL); Amit GRUBER, Haifa (IL); Oded EDRI, Haifa (IL)

(73) Assignees: TECHNION RESEARCH & DEVELOPMENT FOUNDATION LIMITED, Haifa (IL); RAMBAN MEDTECH LTD., Haifa (IL)

(21) Appl. No.: 18/039,381

(22) PCT Filed: Nov. 30, 2021
(86) PCT No.: PCT/IL2021/051424

§ 371 Date: May 30, 2023

Related U.S. Application Data
(60) Provisional application No. 63/119,033, filed on Nov. 30, 2020.

(51) Int. Cl. C12N 5/077; A61P 9/06

(52) U.S. Cl. CPC C12N 5/0657; A61P 9/06; C12N 2510/00; C12N 2529/10

(57) ABSTRACT

The present invention relates to products and methods for modulating cardiac function.

### BACKGROUND OF THE INVENTION

Abnormalities in cardiac action-potential (AP) morphology, as occurs in the long or short QT syndromes (LQTS, SQTS) and additional acquired and inherited cardiac disorders, can cause different arrhythmias.

The QT interval is a measurement made on an electrocardiogram used to assess some of the electrical properties of the heart. It is calculated as the time from the start of the Q wave (beginning of depolarization of the ventricle) to the end of the T wave (end of the repolarization process) and approximates to the time taken from when the cardiac ventricles start to contract to when they finish relaxing. An abnormally long or abnormally short QT interval is associated with an increased risk of developing abnormal heart rhythms and sudden cardiac death. Another important arrhythmogenic risk is increased QT dispersion, manifested by increased ventricular spatial dispersion of the repolarization (dictated also by action-potential duration) properties.

Long QT syndrome (LQTS) is a condition which affects the repolarization of the heart after a heartbeat. It results in an increased risk for the development of different atrial and life-threatening ventricular arrhythmias (irregular heartbeat) which can lead to sudden cardiac death, in otherwise potentially healthy individuals. These episodes can be triggered by exercise or stress. Conditions associated with long QT syndrome may be inherited (mutations in ion channels responsible for the different congenital long QT syndrome types) or acquired in different cardiac disorders (such as heart failure, hypertrophy), due to drug-induced side effects, due to electrolyte abnormalities, and because of several other disorders

Short QT syndrome (SQTS) is a rare inherited arrhythmogenic syndrome, associated with an increased risk of abnormal heart rhythms and sudden cardiac death. It is caused by mutations in genes encoding ion channels that shorten the cardiac action potential and appears to be inherited in an autosomal dominant pattern.

Among the important underlying causes of several types of atrial and ventricular arrhythmias is the presence of abnormal action potential properties (too long, too short, or too heterogeneous throughout the heart).

Beyond the effect on the electrical activity of the heart and the potential for development of arrhythmias, the action potential properties (and specifically action potential duration) may also affect the mechanical properties of the heart (contractility).

Current strategies to treat arrhythmias involve drugs which lack spatial or temporal resolution, affect the heart globally, and therefore are associated with significant side effects including paradoxically the development of more dangerous arrhythmias (pro-arrhythmias). Catheter ablation and surgery result in irreversible damage and are limited to only a minority of patients with arrhythmias and in some cases like atrial fibrillation also not very effective. The use of an implantable cardiac defibrillator while being effective in saving lives is painful and does not solve the underlying problem.

Current implantable devices use electrical stimulations, and they are not used to alter action potential morphology but rather to pace or to give painful shocks to terminate (defibrillation arrhythmia). As such, they cannot treat the underlying cause of arrhythmia in many cases, where the arrhythmia results from abnormalities in action potential properties.

### SUMMARY OF THE INVENTION

Here the inventors show that optogenetic tools can be used to modulate AP properties in a pre-designed and predictable manner, to correct abnormally long or short action-potential durations, to reduce spatial heterogeneities in repolarization, and consequentially to prevent or reverse arrhythmias resulting from a variety of mechanisms including triggered activity and reentry. Healthy-control and patient-specific LQTS and SQTS human induced pluripotent stem cell-derived cardiomyocytes (hiPSC-CMs) were transduced to express the light-sensitive cationic-channel channelorhodopsin-2 (ChR2) or the anionic-selective opsin, ACR2. Depending on illumination timing, light-induced ChR2 activation could induce robust prolongation or mild shortening of AP duration (APD), while ACR2 activation allowed effective APD shortening. By fine-tuning these approaches, the inventors could normalize the pathological AP properties and suppress arrhythmogenicity in the LQTS/SQTS hiPSC-CMs models. In addition, cardiomyocytes' electrical activity could be completely silenced by prolonged optogenetic stimulation. Using an hiPSC-CMs-based tissue model of reentrant cardiac arrhythmias and engineered cells expressing the light-sensitive proteins, the ability of the action-potential modulating optogenetic interventions to prevent or terminate reentrant arrhythmias is demonstrated.

The present invention provides, in one aspect, a method for modulating action potential (AP) properties of cardiac tissue cells of a patient in need thereof, the method comprising the steps of making the membrane potential of the cardiac tissue cells susceptible to light-dependent modulation and illuminating the cardiac tissue cells during the action potential.

In certain embodiments, the method comprises modulating the AP duration (APD) of the cardiac tissue cells.

In certain embodiments, the method comprises elongating the APD of the cardiac tissue cells.

In certain embodiments, the method comprises increasing the contraction function of the cardiac tissue cells.

In certain embodiments, the method comprises silencing the electrical activity of the cardiac tissue cells.

In certain embodiments, the method comprises illuminating the cardiac tissue cells for about 1 second.

In certain embodiments, the method comprises preventing or treating arrhythmia.

In certain embodiments, the method comprises illuminating the cardiac tissue cells for about 80 to about 200 milli-seconds.

In certain embodiments, the method comprises illuminating at least 25% of the cardiac tissue cells.

In certain embodiments, the method comprises depolarizing the cardiac tissue cells.

In certain embodiments, the method comprises illuminating the cardiac tissue cells in phase 2 or phase 3 of the AP.

In certain embodiments, the method comprises shortening the APD of the cardiac tissue cells.

In certain embodiments, the method comprises decreasing the contraction function of the cardiac tissue cells.

In certain embodiments, the method comprises silencing the electrical activity of the cardiac tissue cells.

In certain embodiments, the method comprises illuminating the cardiac tissue cells for about 1 second.

In certain embodiments, the method comprises preventing or treating arrhythmia.

In certain embodiments, the method comprises illuminating the cardiac tissue cells for about 80 to about 200 milli-seconds.

In certain embodiments, the method comprises illuminating at least 50% of the cardiac tissue cells.

In certain embodiments, the method comprises hyperpolarizing the cardiac tissue cells.

In certain embodiments, the method comprises illuminating the cardiac tissue cells in phase 0, phase 1, phase 2, or phase 3 of the AP.

In certain embodiments, the method comprises modulating the AP morphology.

In certain embodiments, the method comprises changing the slope of repolarization of the AP.

In certain embodiments, the method comprises extending the plateau phase of the AP.

In certain embodiments, the method comprises homogenizing electrical APD differences between cells.

In certain embodiments, the method is for preventing or treating Short QT Syndrome (SQTS).

In certain embodiments, the method is for preventing or treating Long QT Syndrome (LQTS).

In certain embodiments, the method is for preventing or treating early-after-depolarizations (EADs) or triggered beats.

In certain embodiments, the method is in vivo or in vitro.

In certain embodiments, the method is for preventing or treating cardiac arrhythmia.

In certain embodiments, the arrhythmia is polymorphic ventricular tachycardia, Torsade-de-Pointe (TdP), or reentrant arrhythmia.

In certain embodiments, the patient is afflicted with a Short QT Syndrome (SQTS).

In certain embodiments, the patient is afflicted with a Long QT Syndrome (LQTS).

In certain embodiments, the patient is afflicted with cardiac tissue action potential duration (APD) or repolarization heterogeneity.

In certain embodiments, making the membrane potential of the cardiac tissue cells susceptible to light-dependent modulation comprises (a) genetically-transforming the cardiac tissue cells with a light-sensitive ion channel, a light-sensitive ion pump, or a light-sensitive signaling receptor, or (b) contacting the cardiac tissue cells with genetically-transformed cells comprising a light-sensitive ion channel, a light-sensitive ion pump, or a light-sensitive signaling receptor.

In certain embodiments, the method comprises expressing a light-sensitive ion channel, a light-sensitive ion pump, or a light-sensitive signaling receptor in the cardiac tissue cells; contacting the cardiac tissue cells with an oligonucleotide construct encoding a light-sensitive ion channel, a light sensitive ion pump, or a light-sensitive signaling receptor; administering genetically-transformed cells to the patients' heart, wherein the genetically-transformed cells comprise a light-sensitive ion channel, a light-sensitive ion pump, or a light-sensitive signaling receptor; or administering genetically-transformed cells to the patients' heart, wherein the genetically-transformed cells comprise an oligonucleotide construct encoding a light-sensitive ion channel, a light-sensitive ion pump, or a light-sensitive signaling receptor.

In certain embodiments, the light-sensitive ion channel, light-sensitive ion pump, or light-sensitive signaling receptor is selected from the group consisting of ChR2, ChR2/H134, ChETA, ChR/T159C, SFO/SSFO, ReaChR, VChR1, Chronos, Chrimson, ChrimsonR, PsChR2, CoChR, CsChR, CheRiff, C1C2, ChIEF, ChEF, ChD, C1V1, iChloC, SwiChRca, GtACR, PsChR1, Phobos, Aurora, Jaws, Halo/NpHR, eNpHR 3.0, Arch, eArch 3.0, ArchT, ArchT 3.0, Mac, eMac 3.0, BLINK-1, and PAC-K.

Further embodiments, features, advantages and the full scope of applicability of the present invention will become apparent from the detailed description and drawings given hereinafter. However, it should be understood that the detailed description, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1. Experimental scheme and functional characterization of ChR2 in the hiPSC-CMs. [a] Experimental outline: patient-specific or healthy-control hiPSCs are differentiated into cardiomyocytes, transduced to express the different light-sensitive proteins, and subjected to patch-clamp or optical imaging analysis. [b] Representative traces from a whole-cell voltage-clamp experiment in the ChR2-expressing hiPSC-CMs. Peak and steady-state light-induced currents are visible. Scale bars indicate 100 pA and 100 ms on the y and x axes respectively. The stimulation protocol (insert) included voltage steps of 1 sec (with the first 500 ms conducted in darkness followed by 500 ms of continuous blue-light illumination) from −80 mV to 60 mV, with 10 mV increments. [c] Current-voltage relationship of the light-induced ChR2 current in the hiPSC-CMs. Mean±SEM of peak and steady state currents are plotted (n=7). [d-e] Representative traces showing the photocurrents evoked in the hiPSC-CMs by light stimuli applied during either the AP's plateau (d) or repolarization (e) phase in AP-clamp experiments. The upper panel shows the voltage AP-clamp protocol, while the lower panel depicts the measured photocurrents (after subtraction of baseline currents obtained at darkness). [f] A scheme depicting the conceptual differences in the type of photocurrents generated by light-induced ChR2 activation during the different AP phases. An optical stimulus will produce a hyperpolarizing current if the Vₘ is more positive than ChR2−Eᵣₑᵥ (as occurs early during the AP). In contrast, a depolarizing current will be generated if Vₘ is more negative than ChR2−Eᵣₑᵥ (light stimulation during the repolarization phase). Scale bar=200 ms.

FIG. 2. Optical modulation of APD in ChR2-expressing hiPSC-CMs. [a] Scheme showing the electrical and optogenetic stimulations protocols used. Every stimulation protocol included 7 consecutive electrically-stimulated APs at darkness before evaluation of the effects of the optogenetic stimuli. Optogenetic stimulation onset was defined as the time interval between the timing of the electrical pacing stimulus and the initiation of illumination. Optogenetic stimulus duration was defined as the duration of the illumination period. [b-d] Light-induced ChR2 activation during the repolarization phase prolongs APD. Whole-cell current-clamp recordings were used to monitor changes in the AP morphology following optogenetic stimulation of the hiPSC-CMs (scale bars in b, d, e represent 20 mV and 200 ms for the Y and X axes respectively). [b] Representative AP traces acquired during darkness (black) and following delivery of optical stimuli (blue) of various durations (dashed lines). Note the tight correlation between the optical stimulus duration and the resulting APD prolongation. [c] A plot depicting the correlation between the timing of the end of the optical stimuli and the resulting APD₈₀ values. Note that both continuous (black circles) and pulsed illumination (grey squares) stimuli resulted an excellent correlation (Pearson's correlation coefficients: 0.99 and 0.99 respectively, n=12). [d] Comparison of the effects of continuous vs. pulsed (20 ms on/30 ms off pulses) illumination. Note the similar effects on APD prolongation. [e-g] Light-induced ChR2 activation during early-stages of the AP shortens APD. [e] Representative AP traces acquired during darkness (black) and following delivery of optical stimuli of various durations during the early-stage of the AP (onset: 20 ms). Note the resulting alteration of AP morphology and APD shortening. [f] Comparison of the effects achieved by varying the duration of the optical stimulus (50 ms, 100 ms, and 150 ms, n=8) on relative APD₈₀ shortening using both continuous (black) and pulsed (grey) stimulation protocols. *p<0.05. **p<0.01. [g] Changes in the measured APD₈₀ values at baseline and following early optogenetic stimulation. Notice the significant (*p<0.05, n=9) APD₈₀ shortening resulting from application of a 100 ms optical stimulus delivered 20 ms after AP initiation. [h] Summary of the bidirectional APD modulating effects achieved by ChR2 light-activation as a function of the timing and duration of the optical stimulus.

FIG. 3. Optogenetic AP modulation in ChR2-expressing LQTS and SQTS hiPSC-CMs. [a-b] Light-induced ChR2 activation during the repolarization phase prolonged the measured APD of SQTS-hiPSC-CMs. Note in the whole-cell recordings, that the shortened APD in the SQTS-hiPSC-CMs could be significantly prolonged by the optogenetic stimuli and that the degree of APD prolongation correlated with the duration of illumination [a]. Application of a 250 ms-long optical stimulus was able to significantly prolong APD₈₀ (n=6, p<0.05) in these cells to levels that were similar to healthy-control hiPSC-CMs (n=10, p=NS). [b] Scale bars in a, c represent 20 mV and 200 ms for the Y and X axes respectively. [c-d] Light-induced ChR2 activation during the AP's earlier stages (phase 2) could shorten the abnormally-long APD of the LQTS-hiPSC-CMs, as depicted in the current-clamp AP recordings [c]. However, the degree of APD₈₀ shortening achieved by the optimal optogenetic stimulation protocol (onset: 40 ms, duration: 100 ms), despite being statistically significant (*p<0.05, n=5), was still not sufficient to reach the APD₈₀ levels of healthy-control hiPSC-CMs (**p<0.01, n=10) [d]. [e] Light-induced ChR2 activation during the early AP phase in the LQTS-hiPSC-CMs could supress EAD formation. (i) Shown are intracellular AP recordings from the LQTS-hiPSC-CMs at baseline (1 Hz electrical pacing), during the application of the illumination protocol (100 ms-long light pulse applied 40 ms after the electrical pacing stimulus for each individual AP) and following termination of illumination. (ii) Higher time-resolution of (i) showing the development of EADs at baseline in some paced beats (in this example in alternating APs). (iii) Suppression of EADs in all APs during application of the optical stimulation protocol (blue). (iv) Resumption of the arrhythmogenic activity following termination of the illumination protocol. Scale bars: 20 mV for the Y axis, 2 and 1 sec for the X axis of (i) and (ii-iv) respectively.

FIG. 4. Optical modulation of APD in ACR2-expressing hiPSC-CMs. [a] Characterizing the response of the recorded intracellular APs to the application of escalating illumination intensities (50 ms-long, onset: 100 ms) in the ACR2-expressing hiPSC-CMs. Scale bars: 20 mV and 200 ms for the Y and X axes respectively. [b] A plot summarizing the changes in the measured APD₈₀ of the ACR2-hiPSc-CMs as function of the optical stimulus' intensity. [c-d] Characterizing the response of the recorded intracellular APs in ACR2-hiPSc-CMs to the timing of the onset of the applied optical stimulus, which was delivered at a fixed intensity (1.3 mW/mm²) and duration (50 ms). Note in both the intracellular AP recordings [c] and in the plot summarizing the resulting effects on APD₈₀ [d], the clear correlation (Pearson correlation coefficient: 0.98, n=5) between the stimulus onset and APD₈₀ shortening, with the earlier timings of onset leading to the shorter APD₈₀ values. [e] Patch-clamp recordings showing robust shortening of the abnormally long APD values in the LQTS-hiPSC-CMs following light-induced activation of ACR2. Notice that the degree of APD shortening inversely correlated with the onset of the optical stimulus (intensity: 1.3 mW/mm², duration: 50 ms), which was initiated at 50, 100, 150, 200, or 250 ms after AP onset. [f] Summary of the results of application of a 50 ms-long stimulus (intensity: 1.3 mW/mm², onset: 100 ms) to the ACR2-expressing LQTS-hiPSC-CMs. Notice the significant shortening of APD₈₀ values (n=9, *p<0.01) in the LQTS-hiPSC-CMs to levels similar to those recorded in healthy-control hiPSC-CMs (n=10, p=NS).

FIG. 5. Optical monitoring of AP properties during optogenetic stimulation. The hiPSC-CMs were loaded with the voltage-sensitive dye FluoVolt, paced at 1 Hz and monitored using the line-scan mode to derive the optical APs. Scale bars: 200 ms. [a-d] Optogenetic prolongation of the recorded optical APs in ChR2-expressing healthy-control [a-b] and SQTS [c-d] hiPSC-CMs. Shown are representative optically-derived AP signals from a healthy-control [a] and a SQTS [c] hiPSC-CM at baseline (during darkness, black tracings) and during optogenetic stimulation (blue tracings). Illumination consisted of a pulsed stimulation protocol (15 ms-on, 25 ms-off), initiated 60 ms after the AP onset with a duration varying between 2 to 10 light pulses (80-400 ms). Note the significant APD prolongation in the recorded optical tracings [a, c] and summary plots [b, d]; resulting in an excellent correlation between the timing of the end of the optical stimuli and the resulting APD₇₀ changes [Pearson correlation coefficients: 0.97 (n=10) and 0.99 (n=12) for healthy-control and SQTS hiPSC-CMs respectively]. [e] Optogenetic shortening of the recorded optical APD in ACR2-expressing healthy-control hiPSC-CMs. Shown are the optical APs recorded at baseline (during darkness, black tracing) and during optogenetic stimulation (blue tracing). The timing of the onset of the 50 ms-long optical stimulus varied between 50 to 200 ms after AP initiation (electrical pacing). Note that the greatest APD shortening was associated with the earliest delivered optogenetic signals. [f] Comparison of the degree of APD shortening (relative changes in APD₇₀ values) induced by ACR2 activation (50 ms-long stimulus, onset-100 ms) in the LQTS vs. healthy-control hiPSC-CM. Note that while APD shortening was robust in both cell types, the degree of shortening was significantly greater (*p<0.05) in the LQTS-hiPSC-CMs (n=8) when compared control cells (n=19). [g] Plots depicting the relationship between the timing of the onset of the optical stimulus (50 ms duration) and the relative degree of APD₇₀ shortening in LQTS-hiPSC-CMs expressing either ChR2 or ACR2. Note the significantly greater APD shortening associated with ACR2 activation (n=7, **p<0.01) as compared to ChR2 activation (n=4).

FIG. 6. Optogenetic protocols to supress cardiomyocyte excitability. [a-b] Whole-cell patch-clamp recordings from either ChR2-expressing [a] or ACR2-expressing [b] hiPSC-CM. Notice how continuous prolonged 1.3 mW/mm² blue-light illumination was able to clamp the membrane potential to either a depolarized (in the case of ChR2, a) or hyperpolarized (in the case of ACR2, b) potential and suppress spontaneous AP generation. Scale-bar: 40 mV. [c] Representative optical AP recording (using voltage-dye imaging), acquired during continuous electrical field stimulation of hiPSC-CMs expressing either ChR2 (top-panel), ACR2 (middle-panel) or eGFP (bottom-panel). Note that prolonged exposure to 1.3 mW/mm² blue-light completely supressed the development of APs in the hiPSC-CMs expressing ChR2 (top-panel) and ACR2 (middle-panel) despite the continuous electrical pacing. In contrast, the same illumination protocol did not affect control eGFP-expressing hiPSC-CMs (bottom-panel). The timing of blue-light illumination is represented in the figures all tracings by the blue background.

FIG. 7. Generation of light-sensitive cardiac tissue models. [a] Generation of in-vitro models of light-sensitive cardiac tissue, in which two kinds of human cells were co-cultured. First, HEK293 cells were virally transduced to express the light-sensitive ion-channel CoChR. The transduced cells were then selected by FACS and antibiotic selection (not illustrated). The engineered HEK293 cells were seeded together with the hiPSC-CMs to form 2- or 3-dimensional tissue. [b] Current-voltage curve, as calculated from whole-cell patch-clamp experiments in the CoChR-HEK293 cells. n=5. [c] Illustration of the 2-dimensional cardiac sheet model. CoChR-HEK293 cells were seeded as a circle of 1 cm in diameter. Then, a second layer of hiPSC-CMs was seeded on top. [d] Confocal laser scanning microscopy imaging. Two-dimensional (left) and three-dimensional reconstructed z-series (right) of a co-culture tissue. hiPSC-CMs is demonstrated as α-actinin positive (red), engineered HEK293 cells are demonstrated as GFP positive (green), Cx43 (white) for the identification of gap junctions and DAPI (blue) for nuclei. Scale bar=20 mm.

FIG. 8. Optical stimulation and synchronization in hiPSC-CCSs. [a] illustration of the EM-CCD based optical mapping system. Arrows represent connection to different light sources, centred at 630 nm and a 488 nm. [b-e] Characterization of optical pacing, n=5. Mean capture rates are presented for diffuse light stimulations at different stimulation frequencies (each 1 ms long, 0.16 mW/mm²) [b], different stimulation durations (at 1 Hz, 0.16 mW/mm²) [c], and different illumination intensities (1 ms duration at 1 Hz) [d]. [e] Characterization of the minimal illumination duration necessary for 90% capture rate at different illumination intensities, at 1 Hz. n=5. [f-g] Characterization of patterned illumination. [f] Illustrations of the different illumination patterns used (upper row), and their corresponding activation maps (lower row. Isochrones of maximum dF/dt timing is 5 ms, scale bar=1 mm). [g] Statistical analysis of the total activation time calculated from the activation maps. n=8, *P<0.05. **P<0.001. ***P<0.0001.

FIG. 9. Optogenetics-based APD modulation at the tissue level. [a] Scheme describing the derivation of the in-vitro co-culture model. The hiPSC-derived cardiomyocyte cell sheets (hiPSC-CCSs) were seeded on top of a monolayer of CoChR-expressing HEK293 cells. [b] Confocal microscopy based 2D (left) and 3D reconstructed z-series (right) immunostainings of the co-cultures. The hiPSC-CMs are identified as α-actinin positive cells (red) and engineered HEK293 cells by their eGFP expression (green). Gap junctions are indicated by the positive Cx43 punctuate immunosignal (white). Nuclei are counterstained with DAPI (blue). [c] Optogenetic-based APD modulation protocol. Both pacing [short-flash (10 ms), black line] and APD [prolonged pulsed stimulus (100 ms), light-blue] modulation stimuli were achieved through diffuse light exposure of the culture. [d] Representative optical APs recordings at baseline (black-tracing) and during applications of the optogenetic APD modulating stimuli at variable durations (blue-tracings). Note the correlation between the optical stimulus duration and the resulting APD prolongation. Scale-bar: 200 ms. [e] Optical mapping derived color-coded APD₈₀ maps acquired at baseline (darkness, left) and during applications of the APD modulating signals (105/225/345 ms). [f] Summary of changes in APD₈₀ values at baseline (darkness) and following applications of the optogenetic stimuli in healthy-control hiPSC-CCSs (n=6). [g-h] Optogenetic-based modulations of APD₈₀ (n=4, g) and the effective refractory period (ERP) (n=5, h) values in the CoChR-SQTS-hiPSC-CCSs co-cultures. Shown are baseline values and the effects of optogenetic stimuli of different durations (*P<0.05. **P<0.01.***P<0.001.****P<0.0001). P<0.01 when comparing ERP values in SQTS vs. healthy-control hiPSC-CCS.

FIG. 10. Dynamic optogenetic-based action potential duration (APD) modulation for prevention of reentrant arrhythmias in the SQTS-hiPSC-CCS model. [a-c] Optogenetic cross-field protocol to induce spiral waves in the SQTS-hiPSC-CCSs. [a-b] Schemes describing the cross-field optogenetic stimulation protocols in both time [a] and space [b]. The co-culture is optogenetically paced using a point stimulation (S1) from the left-side of the culture. When the S1-induced wavefront reaches the center of the tissue, a perpendicular wavefront is delivered by a broad S2 optogenetic-based stimulation wave originating from the top half of the culture (S2). [c] Sequential fluorescent images taken from the dynamic optical-mapping display depicting the process of spiral-wave induction. At t=0 ms, a point optogenetic pacing stimuli (S1) induces a propagation wave traveling from left to right (t=83 ms). When the propagation wave reaches the center of the culture a broad optogenetic premature stimulation (S2) produced a new wavefront traveling perpendicular to the initial wave (t=102 ms). This new wavefront is able to pre-excite already excitable tissue proximal to the traveling S1 wave (146 ms, marked in a blue circle) and initiate a sustained spiral-wave (181-294 ms). [d-f] Optogenetic APD modulation prevents spiral-wave induction. [d-e] Schemes depicting the optogenetic cross-field stimulation and dynamic APD modulation protocols in both time [d] and space [e]. Following application of S1, an APD-modulating illumination pattern was delivered, which was designed to be identical to the shape of the S1-induced propagating activation wavefront and to follow this wavefront with the same CV and a fixed delay of 40 ms. To complete the cross-field stimulation, a perpendicular wavefront was then induced by S2 as described above. [f] Sequential fluorescent images taken from the dynamic optical-mapping display depicting the prevention of the cross-field induced spiral-wave generation by the APD-modulating signal. The prolongation of the tissue wavelength by the APD-modulating signal is marked with a double-headed blue arrow. This resulted in the prevention of the development of reentrant activity following the premature S2 excitation wavefront. [g-h] Effects of changing the APD-modulation signal properties on its anti-arrhythmic properties. [g] Schemes highlighting the different optogenetic APD modulating pattern used and the resulting maximal APD prolongation (in ms) and degree of wavelength prolongation (in mm) for each intervention. [h] Differences in the spiral-wave induction rate with the different APD modulation protocols. The protocol with no APD modulation served as control (n=15 in 5 independent experiments, *P<0.05, **P<0.001, ***P<0.0001).

FIG. 11. Induction of spiral waves in hiPSC-CCSs. [a, b] Description of spiral wave induction by a diffuse S1S2 protocol. [a] Fluorescence time-lapse snapshots of a representative response in the tissue. The timing of the diffuse S1 and S2 stimulation is marked by the light blue rectangles. Scale bar=1 mm. [b] Statistical analysis of the spiral wave induction rate by the different intervals between S1 and S2 (“S2 onset”). n=9. [c-e] Description of spiral wave induction by the cross-field protocol. [c] Illustration of the temporal (i) and spatial (ii) parameters of the cross-field protocol. [d] Fluorescence time-lapse snapshots of a representative cardiac tissue. The upper panel represents the type and timing of the optical stimulations. The lower panel demonstrates the response to the stimulations. Following the S2 stimulation, a new origin of activation (arrowhead) was evoked, as well as conduction block geometry (dashed line). The new direction of propagation (grey arrow) indicates the forming spiral wave. Scale bar=1 mm. [e] Statistical analysis of the spiral wave induction rate by different S2 onsets. n=8.

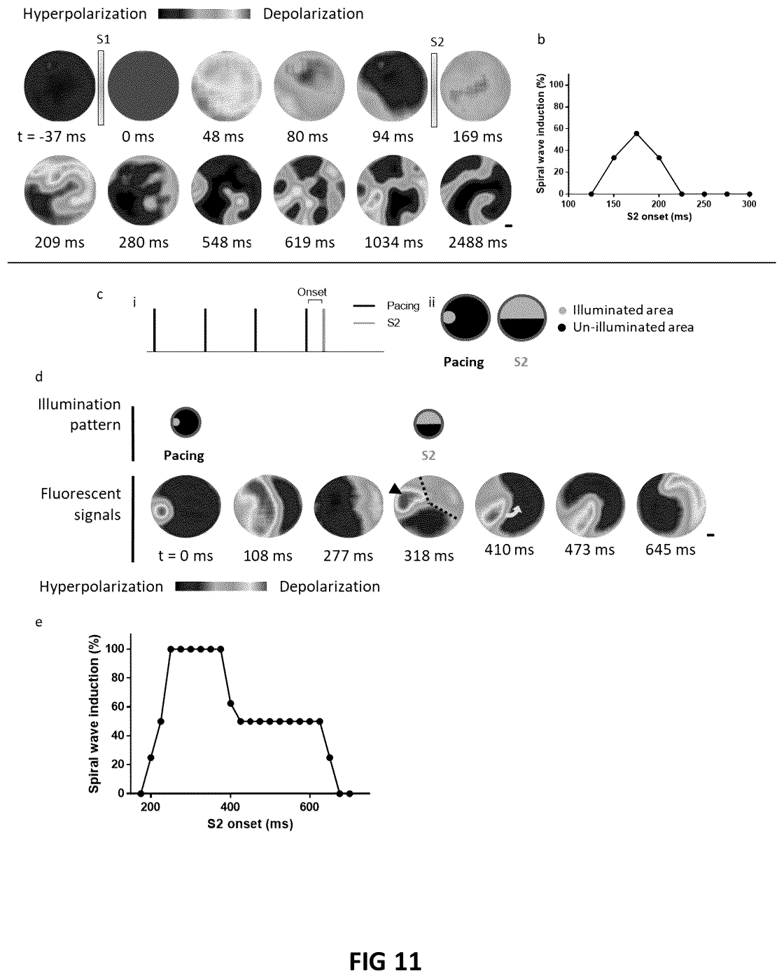

FIG. 12. Spiral wave cessation by optogenetic illumination. [a] Fluorescence time-lapse snapshots of a representative response in a tissue with an ongoing spiral wave activity. The timing of a 20 ms-long diffuse light stimulation is marked by a light blue rectangle. [b] Illustration of the different illumination patterns used. [c] Statistical analysis of spiral wave termination rates with different patterns and durations of illumination, n=15. [d] Statistical comparison among illuminations of 200 ms in different patterns. n=15. P<0.001 in a test for repeated measurements of a binary variable. [e] Statistical comparison between illumination durations of 200 and 400 ms, in a 100% illumination pattern. n=15, *P<0.05.

FIG. 13. Spiral wave cessation by optogenetic illumination in a 3D engineered heart tissue model. [a] Fluorescence time-lapse snapshots of a representative response to a synchronizing diffuse light stimulation in a Channelorhodopsin-2 variant (CheRiff) expressing EHT. The available excitable tissue is stimulated by a 10 ms-long light stimulation (marked by a light blue rectangle), and stops the perpetuating spiral wave. Dashed line indicates the margins of the EHT. Arrows indicate the direction of propagation. Scale bar=1 mm. [b] Statistical presentation of the spiral wave termination rates. The minimal pulse duration of diffuse light for spiral wave termination was characterized. A cumulative success rate is presented based on the finding that in 0-200 ms range of pulse durations an increase in duration does not impair the efficacy of the stimulation. n=10.

FIG. 14. Optical correction of repolarization gradients reduces tissue arrhythmogenicity. [a-b] To generate and model APD heterogeneity in the tissues, healthy hiPSC-CMs were co-cultured with SQTS hiPSC-CMs (a, left and middle panels). Reducing the tissue local repolarization heterogeneity by optogenetic manipulation of the ChR2 expressing tissue. APD dispersion could be diminished by using a diffuse illumination with specific parameters: Onset=40 ms, duration=105 ms (a, right panel, and b). [a] Representative APD maps revealed the areas with different APD80 values (orange and green colored areas, middle panel in a), and demonstrated that APD80 could be homogenized with light (homogenous green color, right panel). ROIs for statistical calculations are marked as black rectangles. [b] Statistical analysis of the APD80 differences in the tissues, calculated as the differences between the mean APD80 values in the ROIs in panel a. Note the significant reduction in APD80 differences following exposure to APD-modulating light. * p<0.01, n=4.

[c-e] Spiral waves evolve in the presence of dispersion of repolarization, and can be prevented with optical APD homogenization (diffuse illumination, Onset=40 ms, duration=105 ms). [c] Illustration of the optogenetic stimulation protocols in time (upper panel) and space (bottom panel). Note the diffuse illumination pattern used for pacing and APD modulation, and the illumination pattern of S2 that covered half of the tissue and was aligned with the direction of the repolarization gradient. [d] Sequential fluorescent images depicting spiral wave formation due to the innate repolarization gradient in the tissue. The timing of the pacing and S2 illuminations is marked with dashed lines. The homogenous activation of the tissue after the pacing, the tissue's innate repolarization gradient, the selective excitation of the recovered tissue within the area that was exposed to S2, and the resulting reentrant waves that evolved (t=0, 118, 367, and 492-2600 ms, respectively). White arrows indicate the direction of propagation. Scale bar=1 mm. [e] Statistical analysis of the spiral wave induction rates by S2 in the presence of an innate gradient of repolarization. Each tissue was examined with S2 onsets of 160, 180 and 200 ms. Note the significant reduction in spiral wave induction rates following treatment with APD-homogenizing illumination with an onset of 40 ms and duration of 105 ms. * p<0.05, n=6.

### DETAILED DESCRIPTION OF THE INVENTION

The cardiac action potential (AP) is generated by a conserved chain of events. The cell membrane selectively changes its conductance to ions on a millisecond time scale. The outcome is a balanced integration of several distinct ionic currents, which greatly influence other important parameters such as the refractory period, conduction velocity, and functional chamber-specification. Consequentially, any pathological changes in the participating currents can disturb AP properties, potentially leading to arrhythmia development and even sudden cardiac death. Such disturbances in the ionic currents balance occur, for example, in the inherited long QT and short QT syndromes (LQTS and SQTS), where loss- or gain-of function mutations alter the biophysical properties of key ionic channels leading to abnormally prolonged or shortened AP duration (APD) respectively, both of which may trigger life-threatening arrhythmias.

Optogenetics refers to the ability to control the activity of excitable cells through the expression of light-sensitive microbial proteins (opsins) functioning as ion channels, ion pumps, or signaling receptors in the targeted cells. In the brain, optogenetics enables targeted modulation of the activity of specific opsin-expressing neuron populations by light and therefore tight spatiotemporal control of neuronal circuitry, completely transforming the field of neuroscience. Similar optogenetic tools were also recently applied to the cardiac arena. These pioneering studies revealed the ability to modulate cardiac tissue electrical activity through light-induced opsins' activation, enabling cardiac optogenetic-based pacing, resynchronization, and even defibrillation. While the focus of cardiac optogenetic strategies has been on inducing or supressing the generation of APs using either depolarizing or hyperpolarizing light-sensitive proteins, the same concepts could potentially also be used to modulate the cardiomyocyte's AP properties, as suggested by computational modelling studies and in proof-of-concept experiments using neonatal rat cardiomyocytes.

Here, this concept is taken a step forward by evaluating the ability to utilize optogenetic tools to shape the AP morphology of human cardiomyocytes and even to correct the abnormal AP properties associated with the LQTS and SQTS. To this end, the inventors used healthy-control and patient-specific human induced pluripotent stem cell derived cardiomyocytes (hiPSC-CMs) that were genetically modified, using lentiviral transduction, to express different light-sensitive channels. By overexpressing the non-selective cationic light-sensitive channel channelorhodopsin-2 (ChR2) in the tested hiPSC-CMs the inventors were able to induce both shortening and robust prolongation of the APD, depending on the illumination timing during the course of the AP. Overexpression of the anionic-selective opsin ACR2 in the hiPSC-CMs allowed for more effective APD shortening of the tested hiPSC-CMs. Using the aforementioned optogenetic strategies, the inventors also revealed the ability to alleviate disease phenotype, normalize the pathological APs, and reduce arrhythmogenicity indices in the patient-specific LQTS and SQTS hiPSC-CMs models. The inventors also demonstrated the ability to completely silence electrical activity in the cardiomyocytes by prolonged optogenetic stimulation.

The AP morphology is an important determinant of physiological cardiac function and when disrupted, as a result of a variety of acquired or inherited causes, it can lead to development of different cardiac arrhythmias. LQTS and SQTS are examples of inherited arrhythmogenic syndromes, where mutation in different ionic current can lead to abnormal prolongation or shortening of the APD duration respectively. If the APD is too long, such as in the case of acquired and inherited long QT syndrome, there is a risk for development of spontaneous EADs and triggered beats, which can cause life-threatening arrhythmias such as the polymorphic ventricular tachycardia Torsade-de-Pointe (TdP). If the APD is too short, on the other hand, the refractory period and the tissue wavelength is shortened; a phenomenon that can increase the risk for the development of reentrant arrhythmias.

In the current studies, the potential of using optogenetic tools to modulate AP properties in human cardiomyocytes and specifically to correct the abnormal AP properties associated with the LQTS and SQTS is evaluated. The inventors initially evaluated the potential of using the light-sensitive cationic channel ChR2, the most abundantly used opsin in optogenetic applications. Similar to its use in neuroscience, where there is no real interest in shaping AP morphology, the primarily use of ChR2 in the cardiac optogenetic field, until recently, was to affect the process of AP generation for applications involving optogenetic-based cardiac pacing, electromechanical resynchronization, or defibrillation. Hence, there is only paucity of experimental data in activating ChR2 during the cardiomyocyte's AP in rat cardiomyocytes and no data with regards to shaping the AP by ChR2 activation in human heart cells (where the plateau phase is more pronounced) and in pathological states.

To evaluate the ability to optogenetically modulate the cardiac AP properties, the inventors utilized healthy-control and patient-specific hiPSC-CMs that were transduced to express channelorhodopsin 2 (ChR2) or its newer modified versions such as CoChR, CheRiff, and ReChR. A carefully-designed diverse optical stimuli was then employed to evaluate the effects of ChR2 activation at different time points during the AP. Surprisingly, the results demonstrate that ChR2 activation can be used bidirectionally, depending on the relative timing of the optogenetic stimulus, producing either depolarizing currents (and thereby prolonging APD) or hyperpolarizing currents (resulting in APD shortening).

These opposing effects result from the relationship between the cardiomyocyte's membrane potential at the time of the optical stimulus relative to the ChR2 reversal potential.

Optical stimulations applied while the membrane potential is more positive than ChR2−Eᵣₑᵥ (early phase 2 of the AP) result in positive (hyperpolarizing) currents, while negative (depolarizing) photocurrents are produced if the membrane potential is more negative than ChR2−Eᵣₑᵥ (phases 3 and 4 of the AP). While using ChR2 to achieve membrane depolarization has been the primary mechanism for its use in the different cardiac optogenetic applications to date, this is the first report describing its potential use also as a hyperpolarizing agent.

The ability to prolong APD in the ChR2-expressing hiPSC-CMs by optical stimuli delivered during phase 3 of the AP was highly robust and predictable, with the resulting APD correlating with the timing of the end of the optogenetic stimulus. This allowed to tailor the specific optical stimulation protocol to achieve any desired APD prolongation. Consequentially, the inventors were able to use this approach to correct the abnormally shortened APD associated with the SQTS and normalize the APD in the SQTS-hiPSC-CMs to levels that did not differ from healthy-control cells.

As discussed, the inventors could also shorten APD in the ChR2-expressing hiPSC-CMs by optical stimuli delivered during early phase 2 of the AP. This effect, however, was relatively limited and the APD could not be shortened beyond a minimal value even when increasing the degree of light intensity or the stimulation duration, probably due to relatively short time window in which Vₘ resides above ChR2−Eᵣₑᵥ. Consequentially, the inventors were able to use this optogenetic approach to significantly shorten the abnormally prolonged APD in the LQTS-hiPSC-CMs, but were not able to completely normalize APD values to the same degree as in the healthy-control hiPSC-CMs. Interestingly, this optogenetic hyperpolarizing effect still seemed to be of therapeutic values, as it was able to completely suppress arrhythmic activity (EADs) in the the LQTS-hiPSC-CMs.

To achieve more significant APD shortening, the inventors sought for alternative light-sensitive proteins, which could produce robust and continuous hyperpolarization effects. Traditional opsins used for suppression of neuronal activity via hyperpolarization include the chloride-pumping rhodopsin NpHR (halorhodospsin) and proton pumps (such as archorhodopsin). Although light-induced activation of both types of opsins was shown to supress, to a certain extent, cardiomyocytes' APs generation in the zebra fish model and in some in-vitro experimental cardiomyocyte models; the effects were rather limited, required high power intensities, could not be used to shape the AP when activated during the repolarization phase, were associated with a rebound effect, and were not reproduced yet in the in-vivo mammalian heart. This limited potency may result from the fact that both aforementioned opsins are pumps, and not an ion channels like ChR2, and therefore produce much smaller photocurrents since they transport only one charge per captured photon.

To overcome this limitation, the inventors examined a new opsin variant, ACR2. ACR2 is part of the recently described anion channelrhodopsins (ACRs) family, which are channelrhodopsins with strict anion selectivity and high unitary conductance. Light-induced ACR activation allowed neuronal inhibition at much lower light intensities than rhodopsin ion pumps and Cl⁻-conducting mutants of cation channelrhodopsins. More recently, ACR expression and light-activation in neonatal rat cardiomyocytes was evaluated and shown to produce hyperpolarizing photocurrents that were markedly higher than those observed following archorhodopsin activation, allowing for significant AP modulation and silencing.

Here the inventors demonstrate the robust ability to use light-activation of ACR2 to shorten APD in hiPSC-CMs. In contrast to the ceiling effect and relatively limited APD shortening achieved by ChR2 activation, the effect induced by ACR activation was robust, unlimited, and could be modulated by parameters such as light-intensity and optical stimulus onset and duration. Consequentially, the degree of APD shortening could be tailored to achieve any desired value. This allowed, for example, to normalize APD duration in the hiPSC-CMs model of the LQTS to levels that were not different from heathy-control hiPSC-CMs.

The ACR2 current is primarily mediated by [Cl⁻] conductance and therefore highly dependent on the intracellular concentration of this anion. Since the whole-cell patch-clamp settings disturbs the cell membrane and also artificially determines the internal ion concentrations, there was a risk of disrupting the natural Cl⁻ concentration inside the cells. The inventors addressed this potential bias by confirming the patch-clamp results with the use of an all-optical system approach in which the studied cells are unperturbed. After loading the cells with the voltage sensitive dye FluoVolt, the inventors could derive optical APs and optically monitor the cells over time during various optogenetic stimulations without perturbing the cells. These studies confirmed that the results of the optogenetic interventions was not dependent on the patch-clamp settings and could be recapitulated in intact cells.

An important technical advancement validated was the ability to use pulsed optogenetic stimulation as an alternative to continuous illumination. This alternative allowed the optical monitoring and accurate reconstruction of optical APs of the cells for the validation studies described above, which was not possible with continuous stimulation due to the significant artifacts created. In addition, the pulsed stimulation alternative can reduce the transmitted light energy.

The inventors next aimed to optogenetically control AP properties also at the tissue level. To this end, the inventors utilized a recently-described large-scale (˜1 cm) circular-shaped hiPSC-derived cardiomyocyte cell sheet (hiPSC-CCS) model and co-cultured the layer of cardiomyocytes on top of a monolayer of genetically-engineered cells (in this example HEK293 cells), engineered to express the potent chanelorhodopsin variant, CoChR. Since HEK293, fibroblasts and other cell types cells which can generate gap-junctions with adjacent cardiomyocytes, CoChR light-activation in the engineered cells is expected to modulate AP properties of the coupled cardiomyocytes through electrotonic interactions. To monitor the electrical activity of the CoChR-HEK293/hiPSC-CCS co-cultures, they were loaded with voltage-sensitive dyes and evaluated with a high-resolution optical mapping system. To test various optogenetic protocols, the inventors utilized a digital micro-mirror device (DMD), allowing the generation of complex spatiotemporal illumination patterns. The protocol used to test optogenetic-based APD modulation included delivery of four consecutive diffuse optogenetic pacing stimuli [10 ms-long flashes] with the last pacing-stimulus immediately followed (onset-120 ms) by the APD-prolonging optogenetic signal. As exemplified in the resulting optical APs traces and APD₈₀ map, light-induced CoChR activation significantly prolonged the tissue's APD, with the degree of APD elongation correlating with optogenetic signal duration. Consequentially, average APD₈₀ values in the CoChR-hiPSC-CCSs increased significantly from a baseline value of 270±31 ms to 328±20, 422±14, and 527±10 ms following illumination durations of 105, 225 or 345 ms respectively.

The inventors next evaluated the ability to optogenetically correct abnormal tissue AP properties by co-culturing CoChR-HEK293 cells with patient-specific SQTS-hiPSC-CCSs. As expected, APD₈₀ values in the SQTS-hiPSC-CCSs were extremely short (98±6 ms) in comparison to healthy-control tissues (270±31 ms). Using the above-mentioned optogenetic APD-modulating protocol, the inventors could diffusely prolong and normalize APD₈₀ values in the SQTS-hiPSC-CCSs. Thus, application of APD-modulating stimuli (illumination-durations: 105, 225, and 345 ms) significantly prolonged the pathological APD₈₀ values in the SQTS-hiPSC-CCS to 168±3, 288±4, and 431±16 ms respectively.

Since a short refractory-period is a key mechanism supporting reentrant arrhythmias in SQTS, the inventors also evaluated the effects of optogenetic APD-modulating protocols on the tissue's effective refractory-period (ERP). The SQTS-hiPSC-CMs exhibited shortened ERP values (170±6 ms) compared to healthy-control tissues (251±18 ms). Using the CoChR-optogenetic APD-modulating signal, the abbreviated ERP in the SQTS-hiPSC-CCSs could be significantly prolonged to 233±7, 283±9 and 326±10 ms using illumination durations of 80, 130, and 180 ms respectively.

Based on the ability to prolong APD and ERP at the tissue level, the inventors next aimed to develop a dynamic APD-modulating optogenetic strategy that could prevent reentrant arrhythmias. To this end, the inventors initially developed a reproducible arrhythmia model using an optogenetic modification of the well-established electrical cross-field stimulation strategy. This optogenetic protocol allowed the generation of reentrant activity in SQTS-hiPSC-CCSs co-cultures in a robust, reproducible, and controlled manner. Specifically, in the SQTS-hiPSC-CCS/CoChR-HEK293 co-culture the inventors initially optogenetically paced the tissue using a point-stimulation (S1) originating from the left-side of the culture. When the S1-derived wavefront reaches the center, a broad perpendicular wavefront is initiated from the top of the culture by an S2 optogenetic stimulus. The S2 propagating wave then impinges on the tale of the S1 activation-wave, initiating spiral-wave reentry. Using this approach, the inventors were able to generate sustained reentry repeatedly in all SQTS-hiPSC-CCSs co-cultures. The inventors next tested the hypothesis that a dynamic APD-modulating optogenetic-based protocol can be formulated that can prevent reentry in the SQTS-hiPSC-CCS model. To this end, the inventors utilized the DMD apparatus to deliver a dynamic optogenetic stimulation protocol, which was patterned to faithfully follow the S1 propagating wavefront in both time (delivered at each pixel with a 40 ms delay after AP onset) and space; propagating in the same direction and with the same conduction-velocity (CV) as the activation wavefront. Application of this dynamic illumination protocol increased both the APD and the resulting tissue wavelength (WL, a product of CV and APD) of the propagating wave. To evaluate whether the aforementioned intervention can prevent arrhythmia-initiation in the SQTS-hiPSC-CCSs, the inventors repeated the same cross-field stimulation protocol but added the dynamic APD-modulating illumination. The APD-prolonging protocol was able to prevent initiation of spiral-wave reentry by S2-activation. This anti-arrhythmic effect resulted from the increase in the size of the refractory tissue at the tail of the S1-propagating wave, preventing re-excitation of this proximal area by the S2-derived activation-wave and consequentially its ability to induce reentry.

The inventors next tested the effects of altering the parameters of the dynamic illumination protocol on its anti-arrhythmic capabilities. Specifically, the inventors compared the arrhythmogenic outcome of the cross-field stimulation protocol that was not supplemented by an APD-modulating illumination protocol (control-group) to 4 protocols that also included dynamic APD-prolonging interventions of various illumination-durations. The results revealed development of spiral-waves following cross-field stimulation in all control (without APD modulation, n=15) experiments. Application of the dynamic APD-modulating protocols significantly reduced the incidence of arrhythmia induction. Interestingly, there was a high correlation between the degree of WL increase and the intervention's anti-arrhythmic potential. For example, while an illumination protocol designed to increase the WL by 1 mm was associated with a 73% incidence (11/15) of spiral-wave induction, increasing the tissue WL by 2.6 mm completely inhibited (0/15) arrhythmia inducibility.

The inventors demonstrated the ability to clamp the membrane potential by optical means (using continuous light activation of either ChR2 or ACR2) and thereby completely supress excitability in the cells during the illumination period. These results were confirmed in both whole-cell patch-clamp recordings and optical monitoring studies, where continuous illumination abolished APs generation despite continuous application of a 1 Hz electrical stimulation protocol.

The patch-clamp studies also provided insights into the different mechanism of optogenetically-based suppression of excitability. In the case of continuous ChR2 activation, the membrane is clamped at a relatively depolarized potential and the inability to induce APs in these cells probably stems from the resulting inactivation of the Na⁺ channels. In contrast, ACR2 activation clamps the membrane to a hyperpolarized potential, preventing the necessary membrane depolarization required to induce APs. These results may have important implications for adding new optogenetic tools (the use of ACR2) and for better mechanistic understanding of potential optogenetic-based therapeutic strategies for tachyarrhythmias; for suppression of automaticity, generation of functional conduction blocks, and defibrillation.

The results provide a robust, functional, and reversible approach to modulate AP properties (including AP prolongation, shortening and silencing) in human cardiomyocytes by optogenetic tools. By utilizing different opsins (ChR2 and ACR2) with unique biophysical properties and applying variable light stimulation patterns the inventors could tailor the procedure to shape AP morphology and derive any desired APD at both the cellular and tissue levels. This allowed the creation of cellular models of repolarization abnormalities, in which LQTS or SQTS like phenotypes could be derived by optogenetic modulation of the AP of healthy-control hiPSC-CMs. Alternatively, the approach also provides potential new therapeutic opportunities for conditions associated with repolarization abnormalities and for arrhythmias in general. This was demonstrated in the study by the ability to correct both the abnormal phenotypes of the LQTS and the SQTS by carefully-planned optogenetic strategies, to prevent the development of reentrant arrhythmias, as well as to terminate reentrant arrhythmias.

There are several direct applications of the approach described here as shown in the experiments: (1) Prevention of cardiac arrhythmias in high risk patients when the action potential duration (APD) is too long (long QT syndrome) or too short (short QT syndrome) or when there is tissue heterogeneity in APD. (2) The ability to prolong APD can be used to increase cardiac contractility (for example for the treatment of heart failure). (3) The ability to shorten APD can be used for localized reduction of contractility to treat hypertrophic cardiomyopathy with left ventricular outlet obstruction. (4) The ability to shorten APD can also be used to reduce ischemic damage during acute ischemia. (5) The ability to reversibly and functionally alter repolarization properties in both space and time can be used to generate in vitro models of arrhythmias to screen the effects of new therapies.

The effect of local dispersion of repolarization on hiPSC-CCSs and its arrhythmogenicity was also investigated. A tissue model comprised of healthy hiPSC-CM and SQT hiPSC-CMs was established. Optogenetics was used to reduce APD gradients within the tissue and to prevent arrhythmia induction within the tissues. The results show that by applying a diffuse illumination over the tissue, the APD gradient within the tissue was reduced, a more homogenous APD was presented, and the arrhythmia induction rate was reduced.

The ability to silence cardiomyocyte electrical activity by continuous light-induced activation of depolarizing or hyperpolarizing coupled with the ability to deliver dynamically complex illumination patterns (in both space and time) is used to develop novel anti-arrhythmic strategies including: (1) a functional approach to dynamically modulate electrical activity with a high spatial (50% or more of the affected area) and temporal resolutions (milliseconds) as to disrupt and therefore terminate cardiac reentrant tachyarrhythmias, (2) a functional approach to dynamically modulate electrical activity with a high spatial and temporal resolution as to prevent cardiac tachyarrhythmias, (3) reduce energy use by optimization illumination pattern to achieve minimal (or optimal) illumination area to terminate arrhythmias (for example, illumination of 15 milliseconds followed by darkness of 25 milliseconds was effective as continuous illumination of 40 milliseconds).

The present invention provides, in one aspect, a method for modulating action potential (AP) properties of cardiac tissue cells of a patient in need thereof, the method comprising the steps of (1) making the membrane potential of the cardiac tissue cells susceptible to light-dependent modulation, and (2) illuminating the cardiac tissue cells during the action potential.

In certain embodiments, the method comprises modulating the AP duration (APD) of the cardiac tissue cells.

In certain embodiments, the method comprises elongating the APD of the cardiac tissue cells.

In certain embodiments, the method comprises increasing the contraction function of the cardiac tissue cells.

In certain embodiments, the method comprises silencing the electrical activity of the cardiac tissue cells.

In certain embodiments, the method comprises illuminating the cardiac tissue cells for at least about 1 second. In certain embodiments, the method comprises illuminating the cardiac tissue cells for about 1 second.

In certain embodiments, the method comprises preventing or treating arrhythmia. In certain embodiments, the method comprises preventing arrhythmia. In certain embodiments, the method comprises treating arrhythmia.

In certain embodiments, the method comprises illuminating the cardiac tissue cells for about 10 to about 2000 milli-seconds. In certain embodiments, the method comprises illuminating the cardiac tissue cells for about 100 to about 200 milli-seconds. In certain embodiments, the method comprises illuminating the cardiac tissue cells for about 150 milli-seconds.

In certain embodiments, the method comprises illuminating at least 25% of the cardiac tissue cells. In certain embodiments, the method comprises illuminating at least 50% of the cardiac tissue cells. In certain embodiments, the method comprises illuminating at least 75% of the cardiac tissue cells. In certain embodiments, the method comprises illuminating at least 80% of the cardiac tissue cells. In certain embodiments, the method comprises illuminating at least 90% of the cardiac tissue cells.

In certain embodiments, the method comprises illuminating about 25% of the cardiac tissue cells. In certain embodiments, the method comprises illuminating about 50% of the cardiac tissue cells. In certain embodiments, the method comprises illuminating about 75% of the cardiac tissue cells. In certain embodiments, the method comprises illuminating about 80% of the cardiac tissue cells. In certain embodiments, the method comprises illuminating about 90% of the cardiac tissue cells. In certain embodiments, the method comprises illuminating about 100% of the cardiac tissue cells.

In certain embodiments, the method comprises depolarizing the cardiac tissue cells.

In certain embodiments, the method comprises illuminating the cardiac tissue cells in phase 2 or phase 3 of the AP. In certain embodiments, the method comprises illuminating the cardiac tissue cells in phase 2 of the AP. In certain embodiments, the method comprises illuminating the cardiac tissue cells in phase 3 of the AP.

In certain embodiments, the method comprises shortening the APD of the cardiac tissue cells.

In certain embodiments, the method comprises decreasing the contraction function of the cardiac tissue cells.

In certain embodiments, the method comprises silencing the electrical activity of the cardiac tissue cells.

In certain embodiments, the method comprises illuminating the cardiac tissue cells for at least 1 second. In certain embodiments, the method comprises illuminating the cardiac tissue cells for about 1 second.

In certain embodiments, the method comprises preventing or treating arrhythmia. In certain embodiments, the method comprises preventing arrhythmia. In certain embodiments, the method comprises treating arrhythmia.

In certain embodiments, the method comprises illuminating the cardiac tissue cells for about 10 to about 2000 milli-seconds. In certain embodiments, the method comprises illuminating the cardiac tissue cells for about 100 to about 200 milli-seconds. In certain embodiments, the method comprises illuminating the cardiac tissue cells for about 150 milli-seconds.

In certain embodiments, the method comprises illuminating at least 25% of the cardiac tissue cells. In certain embodiments, the method comprises illuminating at least 50% of the cardiac tissue cells. In certain embodiments, the method comprises illuminating at least 75% of the cardiac tissue cells. In certain embodiments, the method comprises illuminating at least 80% of the cardiac tissue cells. In certain embodiments, the method comprises illuminating at least 90% of the cardiac tissue cells.

In certain embodiments, the method comprises illuminating about 25% of the cardiac tissue cells. In certain embodiments, the method comprises illuminating about 50% of the cardiac tissue cells. In certain embodiments, the method comprises illuminating about 75% of the cardiac tissue cells. In certain embodiments, the method comprises illuminating about 80% of the cardiac tissue cells. In certain embodiments, the method comprises illuminating about 90% of the cardiac tissue cells. In certain embodiments, the method comprises illuminating about 100% of the cardiac tissue cells.

In certain embodiments, the method comprises hyperpolarizing the cardiac tissue cells.

In certain embodiments, the method comprises illuminating the cardiac tissue cells in phase 0, phase 1, phase 2, or phase 3 of the AP. In certain embodiments, the method comprises illuminating the cardiac tissue cells in phase 0 of the AP. In certain embodiments, the method comprises illuminating the cardiac tissue cells in phase 1 of the AP. In certain embodiments, the method comprises illuminating the cardiac tissue cells in phase 2 of the AP. In certain embodiments, the method comprises illuminating the cardiac tissue cells in phase 3 of the AP.

In certain embodiments, the method comprises modulating the AP morphology.

In certain embodiments, the method comprises changing the slope of repolarization of the AP.

In certain embodiments, the method comprises extending the plateau phase of the AP.

In certain embodiments, the method is for preventing or treating Short QT Syndrome (SQTS). In certain embodiments, the method is for preventing Short QT Syndrome (SQTS). In certain embodiments, the method is for treating Short QT Syndrome (SQTS).

In certain embodiments, the method is for preventing or treating Long QT Syndrome (LQTS). In certain embodiments, the method is for preventing Long QT Syndrome (LQTS). In certain embodiments, the method is for treating Long QT Syndrome (LQTS).

In certain embodiments, the method is for preventing or treating early-after-depolarizations (EADs) or triggered beats. In certain embodiments, the method is for preventing early-after-depolarizations (EADs) or triggered beats. In certain embodiments, the method is for treating early-after-depolarizations (EADs) or triggered beats.

In certain embodiments, the method is for preventing or treating cardiac arrhythmia. In certain embodiments, the method is for preventing cardiac arrhythmia. In certain embodiments, the method is for treating cardiac arrhythmia.

In certain embodiments, the arrhythmia is polymorphic ventricular tachycardia, Torsade-de-Pointe (TdP), or reentrant arrhythmia. In certain embodiments, the arrhythmia is polymorphic ventricular tachycardia. In certain embodiments, the arrhythmia is Torsade-de-Pointe (TdP). In certain embodiments, the arrhythmia is reentrant arrhythmia.

In certain embodiments, the patient is afflicted with a Short QT Syndrom (SQTS).

In certain embodiments, the patient is afflicted with a Long QT Syndrom (LQTS).

In certain embodiments, the patient is afflicted with cardiac tissue action potential duration (APD) or repolarization heterogeneity. In certain embodiments, the patient is afflicted with cardiac tissue action potential duration (APD). In certain embodiments, the patient is afflicted with repolarization heterogeneity.

In certain embodiments, making the membrane potential of the cardiac tissue cells susceptible to light-dependent modulation comprises (a) genetically-transforming the cardiac tissue cells with a light-sensitive ion channel, a light-sensitive ion pump, or a light-sensitive signaling receptor, or (b) contacting the cardiac tissue cells with genetically-transformed cells comprising a light-sensitive ion channel, a light-sensitive ion pump, or a light-sensitive signaling receptor.

In certain embodiments, making the membrane potential of the cardiac tissue cells susceptible to light-dependent modulation comprises genetically-transforming the cardiac tissue cells with a light-sensitive ion channel, a light-sensitive ion pump, or a light-sensitive signaling receptor. In certain embodiments, making the membrane potential of the cardiac tissue cells susceptible to light-dependent modulation comprises contacting the cardiac tissue cells with genetically-transformed cells comprising a light-sensitive ion channel, a light-sensitive ion pump, or a light-sensitive signaling receptor.

In certain embodiments, the method comprises expressing a light-sensitive ion channel, a light-sensitive ion pump, or a light-sensitive signaling receptor in the cardiac tissue cells; contacting the cardiac tissue cells with an oligonucleotide construct encoding a light-sensitive ion channel, a light sensitive ion pump, or a light-sensitive signaling receptor; administering genetically-transformed cells to the patients' heart, wherein the genetically-transformed cells comprise a light-sensitive ion channel, a light-sensitive ion pump, or a light-sensitive signaling receptor; or administering genetically-transformed cells to the patients' heart, wherein the genetically-transformed cells comprise an oligonucleotide construct encoding a light-sensitive ion channel, a light-sensitive ion pump, or a light-sensitive signaling receptor.

In certain embodiments, the method comprises expressing a light-sensitive ion channel, a light-sensitive ion pump, or a light-sensitive signaling receptor in the cardiac tissue cells. In certain embodiments, the method comprises contacting the cardiac tissue cells with an oligonucleotide construct encoding a light-sensitive ion channel, a light sensitive ion pump, or a light-sensitive signaling receptor. In certain embodiments, the method comprises administering genetically-transformed cells to the patients' heart, wherein the genetically-transformed cells comprise a light-sensitive ion channel, a light-sensitive ion pump, or a light-sensitive signaling receptor. In certain embodiments, the method comprises administering genetically-transformed cells to the patients' heart, wherein the genetically-transformed cells comprise an oligonucleotide construct encoding a light-sensitive ion channel, a light-sensitive ion pump, or a light-sensitive signaling receptor.

In certain embodiments, the light-sensitive ion channel, light-sensitive ion pump, or light-sensitive signaling receptor is selected from the group consisting of ChR2, ChR2/H134, ChETA, ChR/T159C, SFO/SSFO, ReaChR, VChR1, Chronos, Chrimson, ChrimsonR, PsChR2, CoChR, CsChR, CheRiff, C1C2, ChIEF, ChEF, ChD, C1V1, iChloC, SwiChRca, GtACR, PsChR1, Phobos, Aurora, Jaws, Halo/NpHR, eNpHR 3.0, Arch, eArch 3.0, ArchT, ArchT 3.0, Mac, and eMac 3.0.

In certain embodiments, the light-sensitive ion channel, light-sensitive ion pump, or light-sensitive signaling receptor is ChR2. In certain embodiments, the light-sensitive ion channel, light-sensitive ion pump, or light-sensitive signaling receptor is ChR2/H134. In certain embodiments, the light-sensitive ion channel, light-sensitive ion pump, or light-sensitive signaling receptor is ChETA. In certain embodiments, the light-sensitive ion channel, light-sensitive ion pump, or light-sensitive signaling receptor is ChR/T159C. In certain embodiments, the light-sensitive ion channel, light-sensitive ion pump, or light-sensitive signaling receptor is SFO/SSFO. In certain embodiments, the light-sensitive ion channel, light-sensitive ion pump, or light-sensitive signaling receptor is ReaChR. In certain embodiments, the light-sensitive ion channel, light-sensitive ion pump, or light-sensitive signaling receptor is VChR1. In certain embodiments, the light-sensitive ion channel, light-sensitive ion pump, or light-sensitive signaling receptor is Chronos. In certain embodiments, the light-sensitive ion channel, light-sensitive ion pump, or light-sensitive signaling receptor is Chrimson. In certain embodiments, the light-sensitive ion channel, light-sensitive ion pump, or light-sensitive signaling receptor is ChrimsonR. In certain embodiments, the light-sensitive ion channel, light-sensitive ion pump, or light-sensitive signaling receptor is PsChR2. In certain embodiments, the light-sensitive ion channel, light-sensitive ion pump, or light-sensitive signaling receptor is CoChR. In certain embodiments, the light-sensitive ion channel, light-sensitive ion pump, or light-sensitive signaling receptor is CsChR. In certain embodiments, the light-sensitive ion channel, light-sensitive ion pump, or light-sensitive signaling receptor is CheRiff. In certain embodiments, the light-sensitive ion channel, light-sensitive ion pump, or light-sensitive signaling receptor is C1C2. In certain embodiments, the light-sensitive ion channel, light-sensitive ion pump, or light-sensitive signaling receptor is ChIEF. In certain embodiments, the light-sensitive ion channel, light-sensitive ion pump, or light-sensitive signaling receptor is ChEF. In certain embodiments, the light-sensitive ion channel, light-sensitive ion pump, or light-sensitive signaling receptor is ChD. In certain embodiments, the light-sensitive ion channel, light-sensitive ion pump, or light-sensitive signaling receptor is C1V1. In certain embodiments, the light-sensitive ion channel, light-sensitive ion pump, or light-sensitive signaling receptor is iChloC. In certain embodiments, the light-sensitive ion channel, light-sensitive ion pump, or light-sensitive signaling receptor is SwiChRca. In certain embodiments, the light-sensitive ion channel, light-sensitive ion pump, or light-sensitive signaling receptor is GtACR. In certain embodiments, the light-sensitive ion channel, light-sensitive ion pump, or light-sensitive signaling receptor is PsChR1. In certain embodiments, the light-sensitive ion channel, light-sensitive ion pump, or light-sensitive signaling receptor is Phobos. In certain embodiments, the light-sensitive ion channel, light-sensitive ion pump, or light-sensitive signaling receptor is Aurora. In certain embodiments, the light-sensitive ion channel, light-sensitive ion pump, or light-sensitive signaling receptor is Jaws. In certain embodiments, the light-sensitive ion channel, light-sensitive ion pump, or light-sensitive signaling receptor is Halo/NpHR. In certain embodiments, the light-sensitive ion channel, light-sensitive ion pump, or light-sensitive signaling receptor is eNpHR 3.0. In certain embodiments, the light-sensitive ion channel, light-sensitive ion pump, or light-sensitive signaling receptor is Arch. In certain embodiments, the light-sensitive ion channel, light-sensitive ion pump, or light-sensitive signaling receptor is eArch 3.0. In certain embodiments, the light-sensitive ion channel, light-sensitive ion pump, or light-sensitive signaling receptor is ArchT. In certain embodiments, the light-sensitive ion channel, light-sensitive ion pump, or light-sensitive signaling receptor is ArchT 3.0. In certain embodiments, the light-sensitive ion channel, light-sensitive ion pump, or light-sensitive signaling receptor is Mac. In certain embodiments, the light-sensitive ion channel, light-sensitive ion pump, or light-sensitive signaling receptor is eMac 3.0. All acronyms are well-known in the field, and can be found e.g. on the “Glossary of Microbial Opsin Variants” on https://www.addgene.org/guides/optogenetics/on November 2020.

A device for modulating action potential (AP) properties of cardiac tissue cells, the device comprising (i) a light-emitting element, wherein the light-emitting element is implantable in humans or bio-compatible with human tissue, (ii) optionally, a power source element, wherein the power source element is functionally linked to the light-emitting element and configured to allow the light-emitting element to emit light, and (iii) optionally, a conduit element, wherein the conduit element is mechanically linked to the light-emitting element and to the power source element.

In certain embodiments, the light-emitting element is implantable in humans and bio-compatible with human tissue

In certain embodiments, the device comprises the light-emitting element and the power source element. In certain embodiments, the device comprises the light-emitting element, the power source element, and the conduit element.

In certain embodiments, the light-emitting element is flexible. In certain embodiments, the conduit element is flexible. In certain embodiments, the light-emitting element is rigid. In certain embodiments, the conduit element is rigid.

In certain embodiments, the power source element is implantable in humans or bio-compatible with human tissue. In certain embodiments, the conduit element is implantable in humans or bio-compatible with human tissue. In certain embodiments, the power source element and the conduit element are implantable in humans or bio-compatible with human tissue. In certain embodiments, the power source element and the conduit element are implantable in humans and bio-compatible with human tissue.

In certain embodiments, the light-emitting element is configured to emit light at 470 nm. In certain embodiments, the light-emitting element is configured to emit light at 488 nm. In certain embodiments, the light-emitting element is configured to emit light at 543 nm. In certain embodiments, the light-emitting element is configured to emit light at 630 nm.

In certain embodiments, the light-emitting element is configured to emit light at an intensity of 0.16 mW/mm². In certain embodiments, the light-emitting element is configured to emit light at an intensity of 1.3 mW/mm².

In certain embodiments, the light-emitting element is configured to emit light pulses of 10 to 2000 milli-seconds. In certain embodiments, the light-emitting element is configured to emit light pulses of 100 to 200 milli-seconds. In certain embodiments, the light-emitting element is configured to emit light pulses of 15 milli-seconds. In certain embodiments, the light-emitting element is configured to emit light pulses of 25 milli-seconds. In certain embodiments, the light-emitting element is configured to emit light pulses of 40 milli-seconds. In certain embodiments, the light-emitting element is configured to emit light pulses of 150 milli-seconds.

In certain embodiments, the light-emitting element emits light in the shape of a point or a dot. In certain embodiments, the light-emitting element emits light in the shape of a line or a linear form.

In certain embodiments, the light-emitting element emits light from a single location. In certain embodiments, the light-emitting element emits light from one of its ends. In certain embodiments, the light-emitting element emits light from a plurality of location. In certain embodiments, the light-emitting element emits from at least 50% of its surface. In certain embodiments, the light-emitting element emits from at least 60% of its surface. In certain embodiments, the light-emitting element emits from at least 70% of its surface. In certain embodiments, the light-emitting element emits from at least 80% of its surface. In certain embodiments, the light-emitting element emits from at least 90% of its surface.

In certain embodiments, the light-emitting element emits light from a surface of 1 to 100 mm². In certain embodiments, the light-emitting element emits light from a surface of 10 to 50 mm².

In certain embodiments, the light-emitting element is an LED lamp or an LED light bulb.

In certain embodiments, the power source element is configured to support light intensity of 0.16 mW/mm². In certain embodiments, the power source element is configured to support light intensity of 1.3 mW/mm².

In certain embodiments, the power source element is configured to provide power pulses of 10 to 2000 milli-seconds. In certain embodiments, the power source element is configured to provide power pulses of 100 to 200 milli-seconds. In certain embodiments, the power source element is configured to provide power pulses of 15 milli-seconds. In certain embodiments, the power source element is configured to provide power pulses of 25 milli-seconds. In certain embodiments, the power source element is configured to provide power pulses of 40 milli-seconds. In certain embodiments, the power source element is configured to provide power pulses of 150 milli-seconds.

In certain embodiments, the light-emitting element is implantable in humans or bio-compatible with human tissue.

In certain embodiments, the light-emitting element is configured to emit light on the cardiac tissue cells while being in contact with the cardiac tissue cells.

In certain embodiments, the light-emitting element is configured to emit light on the cardiac tissue cells without being in contact with the cardiac tissue cells.

In certain embodiments, the light-emitting element is configured to emit light on the cardiac tissue cells via or through a blood vessel, a skin layer, a fat layer, or any combination thereof. In certain embodiments, the light-emitting element is configured to emit light on the cardiac tissue cells via or through a blood vessel. In certain embodiments, the light-emitting element is configured to emit light on the cardiac tissue cells via or through a skin layer. In certain embodiments, the light-emitting element is configured to emit light on the cardiac tissue cells via or through a fat layer.

In certain embodiments, the light-emitting element is a plurality of light-emitting elements. In certain embodiments, at least part of the light-emitting element is configured to be used within the human body. In certain embodiments, the entire light-emitting element is configured to be used within the human body. In certain embodiments, at least part of the light-emitting element is configured to be used outside the human body. In certain embodiments, the entire light-emitting element is configured to be used outside the human body.

### EXAMPLES

### Methods

Propagation of hiPSCs and Cardiomyocyte Differentiation.

Previously established healthy-control, LQTS, and SQTS (unpublished) hiPSC lines were used in his study. The hiPSCs colonies were propagated using mTeSR-1 medium. For cardiomyocyte differentiation, a modification of a monolayer directed differentiation system was used as previously described. In brief, the differentiation RPMI/B27 medium containing RPMI-1640, 2% B27 supplement minus insulin (Life Technologies), 1% penicillin/streptomycin, and 6 mM CHIR99021 (Stemgent) was used for two days. Medium was then changed to RPMI/B27 (without CHIR) and on days 3-4 it was supplemented with 2 μM Wnt-C59 (Selleckchem). The resulting beating monolayers (20-60 days of differentiation) were enzymatically dissociated into small clusters or single cardiomyocytes using TrypLE and then plated on matrigel-coated MatTek plates for the different studies.

Lentiviral Transduction of the ChR2 and the ACR2 Transgenes.

The pLV-CAG-ChR2-GFP, pLV-CAG-ACR2-YFP, and pTK113-GFP plasmids were used for virus production in 80% confluent HEK 293T cells, plated on poly-L-lysine coated 10 cm dish. The relevant plasmid was co-transfected with the packaging cassette NRF and the VSVG plasmid using PolyJet reagent (SignaGen Laboratories). Total amount of 8 μg DNA was used per dish, where the transgene of interest, NRF, and VSVG plasmids were mixed in a 3:2:1 ratio, respectively. Fresh virus-containing media were collected at 48 and 72 h and used for two rounds of infections of dissociated hiPSC-CMs to improve transduction efficiency.

Whole-Cell Patch Clamp Recordings.

Whole-cell recordings were conducted with MultiClamp700B and Digidata1440A (Axon-Instruments). The dissociated hiPSC-CMs were perfused with the bath-solution containing (in mM): NaCl-140, KCl-3, HEPES-10, glucose-10, MgCl2-2, and CaCl2-2 (pH-7.4, adjusted with NaOH) and maintained at 35° C. using an automatic temperature controller (Warner Instruments).

For AP recordings the current-clamp mode was used. The hiPSC-CMs were paced at 1 Hz with a 5-ms suprathreshold current pulse. Each cell was first studied at baseline (darkness) and the same cell was then evaluated while treated by pre-designed time-targeted illumination (470 nm monochromatic light at 1.3 mW/mm²) protocols. To minimize rate-related variability in APD, every stimulation protocol included at least 5 consecutive electrical stimulations at a constant rate before data was used for analysis. Action potential duration was estimated by measuring the time to 80% of repolarization (APD80) using the Clampfit 10.7 software (Molecular-Devices).

To assess the developed ChR2 or ACR2 photocurrents, voltage- and AP-clamp experiments were conducted. The experiments were performed during darkness and under blue-light illumination and the elicited photocurrents were calculated by subtracting the former measurement from the latter. The resulting current-voltage (I-V) curve describing both the peak and the steady-state photocurrents was constructed for membrane potentials ranging from −80 mV to 60 mV at 10 mV increments. For AP-clamp studies, pre-recorded AP waveforms served as voltage commands.

Optical Monitoring of hiPSC-CMs.

Optical monitoring was performed 5-7 days after lentiviral transduction of the hiPSC-CMs to express the ChR2 or ACR2 transgenes. Cells were loaded with the voltage-sensitive dye FluoVolt (FluoVolt™ Membrane Potential Kit, Invitrogen) for 30 minutes at 37° C., and then washed and replaced by extracellular Tyrode's solution containing (in mM): NaCl 140, KCl 3, HEPES 10, glucose 10, MgCl2 2, and CaCl2 2 (pH 7.4, adjusted with NaOH). The experiments were conducted at a steady temperature of 37° C. A laser scanning confocal microscope (LSM710, Zeiss) was used to acquire the optical APs by monitoring the fluorescent intensity of FluoVolt in the studied cells. To this end, the inventors excited selected lines on the cell membrane with a 543 nm laser, and monitored the emission through a BP620/52 filter (Chroma Technology Corp.).

Optical experiments were performed on healthy-control and SQTS hiPSC-CMs during 1 Hz electrical pacing and on LQTS-hiPSC-CMs during 0.5 Hz electrical pacing induced by two field-stimulation electrodes. Each cell was paced for at least 5 seconds before optical stimulation was applied, to reduce rate-related variability. Optical stimulations were applied at 1.3 mW/mm². The photo-multiplier tubes (PMTs) in use were sensitive enough to detect the blue light pulses, so a custom-made Matlab software was used to eliminate light artifacts by removing the data acquired during the exposure to blue light by a threshold-based algorithm, and filling the gaps in the optical traces by Matlab ‘fillgaps’ algorithm. To estimate APD, the inventors measured APD70 instead of APD80 due to the lower signal-to-noise ratio of the optical signals. APD70 during optical stimulation was then paired and compared to APD70 from the same cell, acquired at baseline during darkness.

Optogenetics Illumination.

Optogenetics illumination for the single-cell experiments was performed using a 470 nm fiber-coupled LED connected to high-power LED-driver (Thorlabs). A programmable stimulus-generator (STG-1004, multichannels systems) was used to control illumination timing, duration and intensity and to couple its delivery to the electrical stimulus used for AP generation. The onset of optogenetic stimulation was defined as the time-interval between the electrical pacing stimulus and beginning of optogenetic stimulation. For experiments at the tissue level (hiPSC-CCSs), illumination patterns were generated by a digital micro-mirror device (DMD, Polygon-400, Mightex systems) controlled by PolyScan software. Illumination was pulsed (9 ms on/15 ms off).

Establishing the Engineered HEK293 Cells.

CoChR-expressing HEK293 cells were established using lentiviral transduction of the CoChR-GFP transgene. Cells were sorted based on eGFP-fluorescense using FACSAria (BD-Biocsiences) and further purified using a 10-day G418 antibiotic (800 μg/ml) selection process.

Establishing the CoChR-HEK293/hiPSC-CCSs Co-Culture Models.

180,000 CoChR-HEK293 cells were seeded in the 10 mm inner-well of Matrigel-coated MatTet plate (P35G-1.5-10-C, MatTek) as a monolayer. 24 h later, seeded cells were treated with 24 μM Mitomycin-C(Sigma) for 1 h to prevent cell-proliferation. At day-2, 1×10⁶ hiPSC-CMs (either control or SQTS) were seeded on top of the CoChR-HEK monolayer to generate the co-cultures. The culture medium was supplemented with 5 μM Blebbistatin (B0560-5MG, Sigma) to prevent vigorous contraction

Optical Mapping of the hiPSC-CCSs Co-Cultures.

Co-cultures were loaded with the voltage-sensitive dye Di4-ANBDQBS (22.5 μg/ml, acquired from Leslie Loew, University of Connecticut) for 15 min at room temperature. Optical mapping was performed using an EM-CCD (Evolve-512 Delta, Photometrics) and a macroscope (Olympus MVX10). The X-Cite Turbo LED-system served as light source. Excitation filter for Di4-ANBDQBS was Chroma ET620/60x and emission filter was Chroma ET6651p. Micro-Manager software was used for acquisition, and the OMProCCD software, a custom-made IDL-based software (generously provided by Prof. Burn-Rak Choi, Brown University) served for analysis.

To analyze the data, light artifact from the optogenetic stimulation were eliminated and replaced by a threshold-based custom-made Matlab algorithm. Optical signals at each pixel were then analyzed to measure the local activation time (timing of the maximal dF/dt value) and APD₈₀ (the time difference between maximal dF/dt and 80% decay from peak to baseline). These values were used to generate detailed activation and APD₈₀ maps.

Statistical Analysis.

Statistical analysis was performed using GraphPad Prism software. Data was presented as mean±SEM. Differences between groups were compared using unpaired student t-test. For studies comparing measurements from the same cells at baseline (darkness) and following optogenetic illuminations, values were compared using paired student t-test. For studies involving multiple comparisons of one or two independent variables, either one-way or two-way ANOVA was performed respectively, followed by post-hoc Tukey test. To evaluate potential correlation between the optogenetic stimulation parameters and the resulting changes in APD values, the Pearson correlation coefficient was calculated along with two-tailed p-value and a regression model. A value of p<0.05 was considered statistically significant.

### Example 1. Expression and Functional Characterization of ChR2 in the hiPSC-CMs

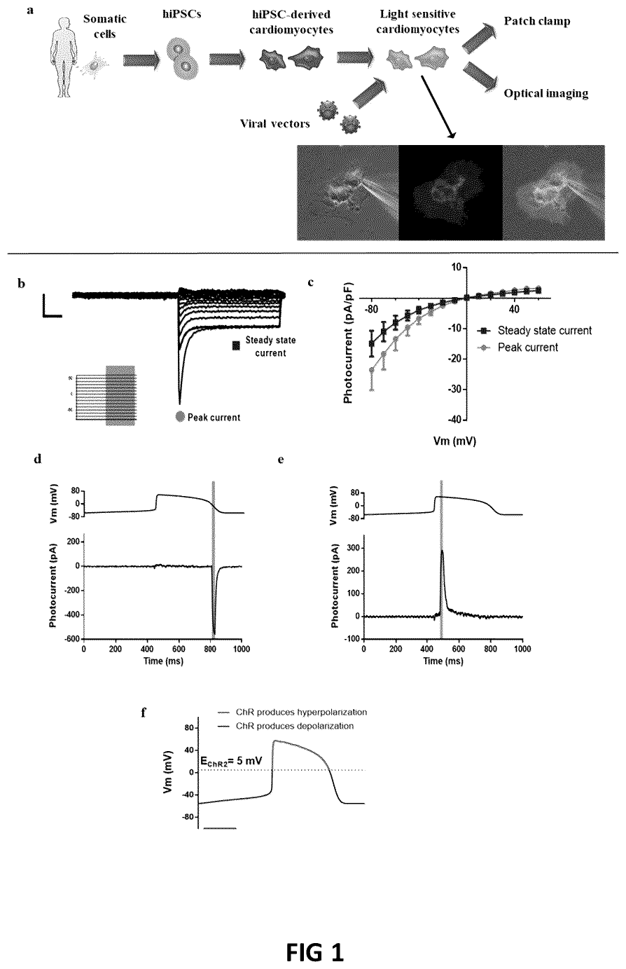

Cardiomyocyte differentiation of healthy-control hiPSC line was achieved using a monolayer based directed differentiation system. The generated hiPSC-CMs were then dispersed into single cells by enzymatic dissociation and plated on Matrigel-coated coverslips. Lentiviral transduction was used to deliver ChR2-GFP transgene to the plated cells. The transduced hiPSC-CMs (identified by their eGFP fluorescence, FIG. 1a) were then evaluated by detailed whole-cell patch-clamp experiments or by optical imaging studies (FIG. 1a).

Whole-cell voltage-clamp experiments, which were performed during darkness and under blue-light illumination, revealed the presence of a robust light-sensitive current in the ChR2-expressing hiPSC-CMs (FIGS. 1b-c). In the experimental protocol used (FIG. 1b), each voltage step lasted 1 second, with the first 500 ms conducted in darkness followed by 500 ms of continuous blue-light illumination (470 nm monochromatic light at 1.3 mW/mm²). A second data set was then acquired during darkness, which was then subtracted from the illuminated recordings to isolate the light-sensitive currents. The resulting current-voltage (I-V) curve, describing both peak and steady-state photocurrents, was constructed for membrane potentials ranging from −80 mV to 60 mV at 10 mV increments (FIG. 1c). The typical known inward rectification properties of the ChR2 current with a reversal potential (ChR2−E_{Rev}) of ˜0 mV is noticed.

Next aimed was the ability to produce light-induced currents during the course of the action-potential (AP) by pre-designed time-targeted illumination protocol. To this end, an AP-clamp experiment in the hiPSC-CMs was designed, and timed the delivery of the optical stimulus to specific time-points during the AP. Specifically, the delivery of an optical stimulus during the repolarization phase (phase 3) was compared when the membrane potential (Vₘ) becomes relatively negative (FIG. 1d) to blue-light delivery during the early plateau phase (early phase 2) when Vₘ is relatively positive (FIG. 1e). As expected, light-induced ChR2 activation during phase 3 of the AP resulted in a robust inward current (FIG. 1d) in the hiPSC-CMs. In contrast, optical stimulation produced a hyperpolarizing photocurrent when illumination was delivered just after the AP's peak (FIG. 1e). The mechanism underlying this significant difference stems from the relationship between ChR2−E_{Rev} and Vₘ as outlined in the scheme in FIG. 1f. Hence, when the Vₘ is relatively hyperpolarized (below ChR2−E_{Rev}), as occurs during late repolarization, an optical stimulus will result in an inward (depolarizing) current. In contrast, an optical stimulation will produce an outward (hyperpolarizing) current if the Vₘ is more positive than the ChR2−Eᵣₑᵥ as occurs during the early stages of the AP.

### Example 2. Optogenetic Modulation of AP Morphology in the ChR2-Expressing hiPSC-CMs

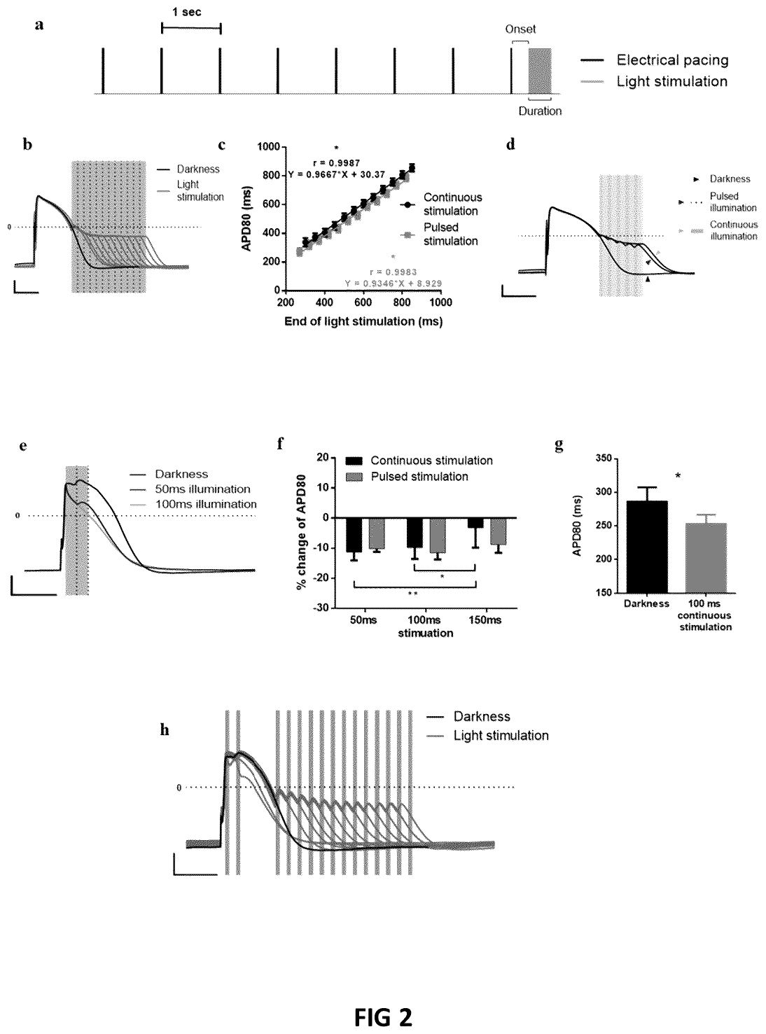

Following demonstration of the ability to produce robust photocurrents in the hiPSC-CMs by targeted illumination and the realization that the nature of these currents (depolarizing or hyperpolarizing) are dependent on the timing of light application, the generated ChR2 photocurrents were utilized to modulate the cardiomyocyte's AP. To this end, the inventors used the current-clamp mode to record APs from the tested hiPSC-CMs. The cells were stimulated at 1 Hz for 7 sec at darkness and then subjected to different illumination protocols during the AP (FIG. 2a).

Initially, the timing of light stimulation was synchronized to phase 3 of the AP in order to trigger depolarizing currents. Specifically, the effects of varying the duration of the optical impulse (starting at 50 ms and prolonging it by 50 ms increments, FIGS. 2a, 2b, 2c) was tested. These experiments revealed the ability of ChR2 light-induced activation to produce sufficient depolarizing currents to prolong the action potential duration (APD) (FIG. 2b). Hence, the resulting APD₈₀ of the treated hiPSC-CM significantly correlated with the timing of the end of the light stimulation protocol (Pearson's correlation coefficient: 0.99, n=12, FIG. 2c). The slope of the resulting linear regression model was ˜1, suggesting that any given time increment in the light stimulation duration resulted in equal increment in the APD₈₀ (FIG. 2c).

Whether the impulse delivered to prolong the AP requires continuous illumination or, alternatively, could include a prolonged pulsed stimulation protocol was also evaluated. To this end, the effects of continuous stimulation to a protocol comprised of alternating 20 ms of light exposure with 30 ms of darkness (FIG. 2d) was compared. This pulsed stimulation protocol resulted in comparable APD prolongations to that achieved with continuous illumination (FIGS. 2c, 2d), with the degree of APD₈₀ prolongation also highly correlating with the timing of the end of the optical stimulus (Pearson's correlation coefficient: 0.99, n=12, FIG. 2c). The importance of the pulsed-protocol is two-fold as it can be designed to allow better optical monitoring (using voltage-sensitive dyes) of the cell's membrane potential (reducing the light-stimulation artifact) and potentially also reduce the energy requirements for this optogenetic strategy.

Following demonstration of successful prolongation of the APD, the hypothesis that similar ChR2 activation can also be used to shorten the APD if the optical stimulus is delivered early during the AP was tested. This hypothesis is based on the results described above (FIG. 1e) showing the development of hyperpolarizing (rather than depolarizing) photocurrents when the illumination is delivered at Vₘ values that are more positive than the ChR2 reversal-potential.

To test the aforementioned hypothesis, the timing of light stimulation to the AP's early-stages, immediately after its peak (FIG. 2e), was synchronized. Illumination was initiated 20 ms after the electrical stimulus inducing the AP, and the tested optical stimuli durations were 50 ms, 100 ms and 150 ms. As shown in FIG. 2e, these illumination protocols significantly altered AP morphology and shortened APD. Hence, both continuous and pulsed (20 ms-on, 30 ms-off) illumination resulted in significant shortening of APD₈₀ by ˜10-15% (FIGS. 2e-f). For example, at 100 ms continuous illumination, APD₈₀ was significantly shortened from 286±21 ms to 253±14 ms (p<0.05, n=9, FIG. 2g).

The above-mentioned results, summarized in FIG. 2h, highlight how light-induced activation of ChR2 can be utilized to bi-directionally affect the AP, either prolonging or shortening APD depending on the exact timing of light stimulation.

### Example 3. Optogenetic-Based AP Modulation in LQTS and SQTS Patient-Specific hiPSC-CMs

The next goal was to use the optogenetic strategies described above to also modulate AP properties in pathological states such as in the long QT (LQTS) and short QT (SQTS) syndromes. To this end, patient-specific hiPSC-CMs models established in the laboratory of the LQTS type 2 (LQTS2) and the SQTS type 1 (SQTS1) was utilized. In LQTS2, loss-of-function KCNH2 mutations reduce the rapid component of the delayed rectifier potassium current (I_{Kr}) thereby prolonging APD. In SQTS1, gain-of-function mutations in the same gene augments I_{Kr}, leading to APD shortening.

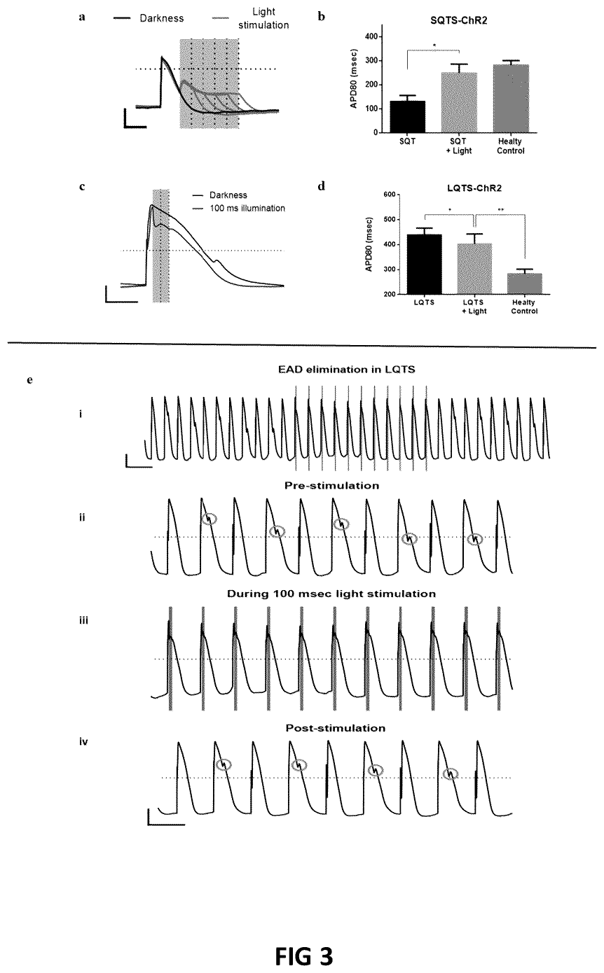

To test the ability of light-induced ChR2 photocurrents to modulate the AP in the LQTS and SQTS, lentiviral transduction to express the ChR2 transgene in the patient-specific hiPSC-CMs was used. The transduced cells were then studied in a similar manner to the initial studies in the healthy-control hiPSC-CMs described above. First focus was on the SQTS, testing the ability to prolong the abnormally short AP of the SQTS-hiPSC-CMs by optogenetic activation of ChR2 during the repolarization phase (FIG. 3a). Similar to the experiments in the healthy hiPSC-CMs (FIGS. 2b-d), the APD in the SQTS-hiPSC-CMs could be prolonged by optogenetic stimulation, with the degree of prolongation correlating with the duration of the optical signal (FIG. 3a). There was no limit to the degree of possible APD₈₀ prolongations and one could tailor a specific optical stimulation protocol to achieve any desired APD value. For example, it was possible to significantly prolong (p<0.05) and correct the abnormally shortened baseline APD₈₀ values (132±25 ms, n=6) in the SQTS-hiPSC-CMs by application of 250 ms continuous illumination to levels (250±37 ms) that were similar to those observed in healthy hiPSC-CMs (283±19 ms, n=10, p=NS) (FIG. 3b).

The hypothesis that light-induced ChR2-activation can also be used to shorten the abnormally long APD in the LQTS-hiPSC-CMs was tested next. This hypothesis was based on the above-mentioned observations where activation of ChR2 during the early stages of the AP could induce hyperpolarizing currents (FIG. 1e) and shorten APD (FIGS. 2e, 2g) in healthy-control hiPSC-CM. As shown in FIG. 3c, application of similar illumination protocols during the early stages of the AP (40 ms after the electrical pacing stimulus) could also shorten the APD of the LQTS-hiPSC-CMs. For example, application of a continuous, 100 ms-long, optical stimulus significantly shortened APD₈₀ of the LQTS-hiPSC-CMs when compared to baseline values during darkness (p<0.05, n=5, FIG. 3d).

In contrast to the APD modulation studies in the SQTS-hiPSC-CMs, where there was no limit on the degree of APD prolongation, in the case of APD shortening in the LQTS-hiPSC-CMs there was a prominent limitation on the degree of the effects achieved. Hence, any increases in the degree of light intensity or in the stimulation duration (beyond 100 ms) were not able to induce further shortening of the APD₈₀ by ChR2 activation. This ceiling effect is probably the result of the limited time-frame in which the membrane potential reside above the ChR2 reversal potential, limiting the duration of the hyperpolarizing effect of ChR2 activation. Consequentially, although optogenetic-based ChR2 activation was able to induce significant APD shortening in the LQTS-hiPSC-CMs (p<0.05, n=5, FIG. 3d), it was not sufficient to shorten APD₈₀ values to levels similar to those measured in healthy-control hiPSC-CMs (p<0.01, n=10, FIG. 3d).

Despite the less-than-optimal optogenetic effect, whether the observed APD shortening could still lead to a functional anti-arrhythmic benefit was evaluated. To this end, the focus was on studying LQTS-hiPSC-CMs that were characterized, in addition to marked AP prolongation, also by the development of early-after-depolarizations (EADs), the harbinger of cardiac arrhythmias in this syndrome. Such LQTS-hiPSC-CMs displaying EADs during continuous 1 Hz electrical pacing (FIG. 3e, i-ii) were then subjected to a 100 ms-long optical stimulus, applied 40 ms after the electrical impulse in each AP (FIG. 3e, i,iii). Impressively, these optogenetic stimuli were able to completely supress the development of EADs in the treated LQTS-hiPSC-CMs (FIG. 3e, iii). EADs re-appeared immediately after cessation of light stimulation (FIG. 3e, iv).

### Example 4. Optogenetic Modulation of the AP Using Anion Channel Rhodopsin 2 (ACR2)

Given the relatively limited ability of ChR2 to shorten the APD, an alternative approach was sought. It was hypothesized that another opsin variant, termed anion channel rhodopsin 2 (ACR2), could produce more potent hyperpolarizing effects due to its ion selectivity properties. While ChR2 conducts cations, ACR2 is an anion selective channel, primarily conducting chloride anions [Cl⁻]. The amplitude of the ACR2 photocurrents and their nature (inward or outward leading to hyperpolarization or depolarization, respectively) depends, among other factors, on the relationship between Vₘ and the channel's reversal potential (ACR−Eᵣₑᵥ).

To provide further insights into the nature of the light-induced ACR current, patch-clamp experiments in HEK cells, transfected to express the ACR2 channel, were conducted. These studies confirmed the importance of the [Cl⁻] gradient in determining the resulting photocurrents and the ACR2 reversal potential (ACR−E_{Rev}). Hence, voltage-clamp studies, conducted at a fixed extracellular [Cl⁻] concentration (151 mM) with varying intracellular [Cl⁻] levels (achieved by changing Cl⁻ concentration in the pipette solution to 151 mM, 30 mM, or 4 mM), revealed different light-induced ACR2 current-voltage curve profiles for each intracellular [Cl⁻] concentration. Consequentially, the calculated ACR2−Eᵣₑᵥ values were also different for each [Cl⁻] concentration [0 mV, −38 mV and −85 mV for [Cl⁻] concentrations of 151 mM, 30 mM, and 4 mM, respectively].

Although the intracellular [Cl⁻] levels in different cell types may differ and are difficult to measure, they are always lower than the extracellular [Cl⁻] level, resulting in a ACR2−Eᵣₑᵥ values that are always negative, as highlighted also in the three experimental scenarios tested. Consequentially, light-induced ACR2 activation should lead to positive electromotive forces (EMF=Vₘ−Eᵣₑᵥ) throughout most of the cardiomyocyte's AP, resulting in robust hyperpolarizing photocurrents.

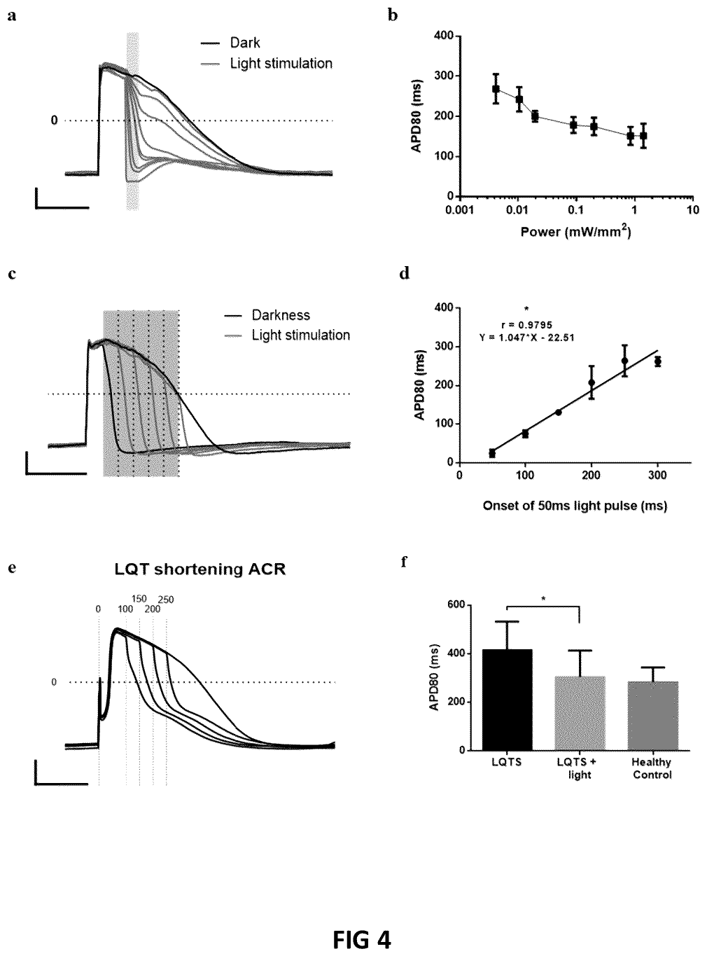

To confirm the aforementioned assumption and to test the ability of utilizing light-induced ACR2 photocurrents to modulate the AP properties, lentiviral transduction to express the ACR2 transgene in the hiPSC-CMs was used. Next, the ability to shorten APD of healthy hiPSC-CM in whole-cell patch-clamp experiments by light-induced ACR activation was tested. An intracellular Cl⁻ concentration of 4 mM was used, a value previously reported in studies in neonatal rat cardiomyocytes. The hiPSC-CMs were electrically paced at 1 Hz and were then subjected to different light-stimulation protocols (FIGS. 4a, 4c). All of these protocols, which were applied during early phase 2 of the AP, resulted in significant alteration of AP morphology and APD shortening (FIGS. 4a, 4c).

Next, the effects of varying light intensity on the resulting APD₈₀ shortening was characterized. To this end, a 50 ms-long optical stimulus (initiated 100 ms after the electrical pacing stimulus) at different intensities was delivered. As noted in FIGS. 4a-4b, an increase in light intensity resulted in a greater degree of APD shortening primarily at low intensity values, whereas at higher values, further increasing light intensity only minimally affected APD.

In further experiments, how the timing of the onset of light stimulation can affect the degree of APD shortening was tested. These studies, utilizing an optogenetic impulse with a fixed intensity (1.3 mW/mm²) and duration (50 ms), revealed that earlier application of the optical stimulus results in a greater degree of APD₈₀ shortening (FIGS. 4c-4d). Consequentially, a linear correlation was found (Pearson's correlation coefficient: 0.98, N=5) between the timing of the onset of the optical stimulus and the resulting APD value, suggesting the ability to reliably control the cardiomyocyte's APD by carefully-planned optical stimulation.

Given the robust and controlled ability to shorten APD in healthy cardiomyocyte, it was hypothesized that a similar approach can be used to shorten and normalize APD also in the pathological state of the long QT syndrome (LQTS). To this end, the ACR2 transgene in the LQTS2-hiPSC-CMs and was expressed, and then a series of optogenetic stimulation studies demonstrating significant shortening of the abnormally long APD of the LQTS-hiPSC-CMs through ACR2 light-activation (FIG. 4e) was performed. The degree of APD shortening inversely correlated with the onset of the optical stimulus allowing to tailor the procedure to achieve the desired results. (FIG. 4e). Consequentially, unlike the limited ability of ChR2 to shorten APD₈₀, optogenetic stimulation of ACR2 (100 ms after the electrical pacing stimulus with a duration of 50 ms and intensity of 1.3 mV/mm²) was able to shorten the APD₈₀ of the LQTS-hiPSC-CMs (from 414±39 ms to 304±36 ms, n=9, p<0.01) to a degree that was similar to healthy-control hiPSC-CMs (283±19 ms, n=10) (FIG. 4f).

### Example 5. Optical Monitoring of the AP Properties During Optogenetic Stimulation

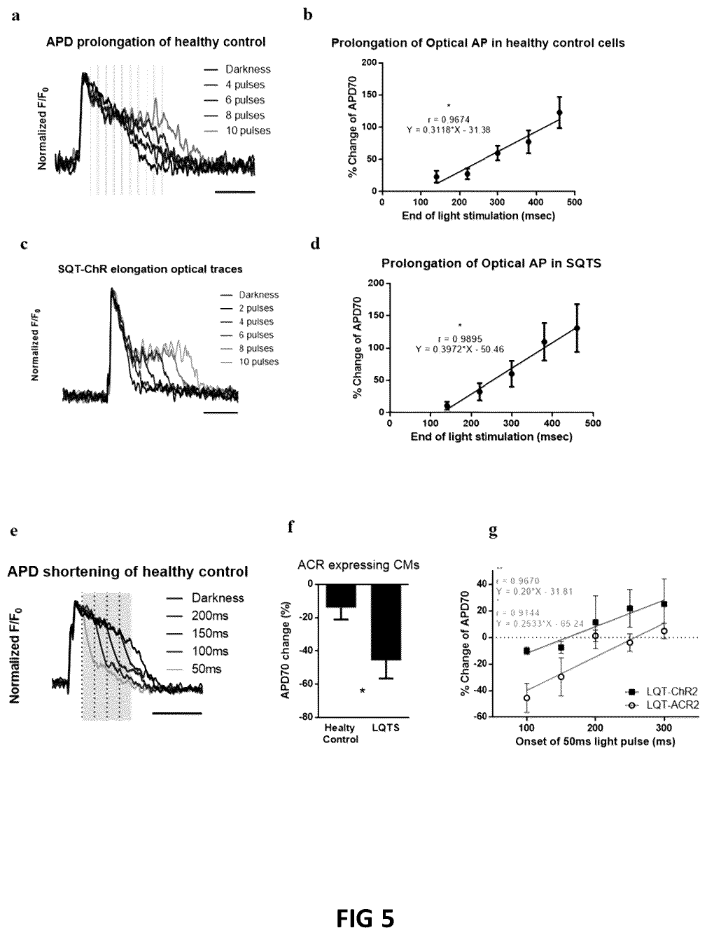

Since patch-clamp is an invasive procedure that allows only short-term and low-throughput studies and may also interfere with the intracellular Cl⁻ concentration, some of the key experiments were repeated using a non-invasive optical method of monitoring the AP that does not perturb the cell membrane. To this end, the hiPSC-CMs, expressing the different light-sensitive proteins, were loaded with the voltage-sensitive dye FluoVolt. The line-scan mode of a laser-scanning confocal microscope was then used to derive optical APs from the tested cells, which were paced at 1 Hz, at baseline and following the application of the different optogenetic protocols (FIG. 5).

To prolong the optical AP, the light-activation of ChR2 in the hiPSC-CMs using a pulsed stimulation protocol (15 ms-on, 25 ms-off) was first evaluated. The duration of the optical activation period, which was initiated 60 ms after the onset of the AP (electrical pacing stimulus), varied between 2 to 10 light pulses (corresponding to 80-400 ms, FIG. 5a). Similar to the patch-clamp experiments, this illumination protocol resulted in significant prolongation of the APD (FIG. 5a) with the timing of the end of light-stimulations correlating with the degree of the optical APD₇₀ prolongation (Pearson correlation coefficient: 0.97, n=10, FIG. 5b). To test the ability to prolong the optical APD also in the pathological state, a similar pulsed light stimulation protocols to activate ChR2 in ChR2-expressing SQTS1-hiPSC-CMs (FIG. 5c) was used. This resulted in significant prolongation of the optical APD₇₀ in the SQTS-hiPSC-CMs (FIG. 5c) with the degree of APD₇₀ prolongation also correlating with the timing of the end of the light stimulation protocol (Pearson correlation coefficient: 0.99, n=12, FIG. 5d).

To evaluate the ability to shorten the optical AP, healthy-control hiPSC-CM were transduced with the ACR2 transgene and then exposed them to light activation (50 ms duration), initiated either 50, 100, 150, or 200 ms after the AP onset. The results in all of these studies was significant APD shortening (FIG. 5e). In a similar manner, light-induced activation of ACR2 using a similar protocol was also able to shorten the APD of the LQTS-hiPSC-CMs, with the relative degree of APD shortening being greater in the LQTS cells. For example, APD was shortened by ˜14% in the healthy-control hiPSC-CMs and by ˜46% in the LQTS-hiPSC-CMs with a 50 ms impulse delivered 100 ms after AP onset (p<0.05, FIG. 5f). The degree of optical APD₇₀ shortening in the LQTS-hiPSC-CMs inversely correlated with the timing of the onset of ACR light-activation, with earlier light applications resulting in greater APD shortening (Pearson correlation coefficient: 0.91, n=7, FIG. 5g). Similar to the patch-clamp experiments, light-induced activation of ChR2-expressing LQTS-hiPSC-CMs during the earliest stages of the APs was also able to shorten the optical APD₇₀, but the degree of shortening was much smaller than that achieved by ACR activation (p<0.01, FIG. 5g).

### Example 6. Continuous Illumination Clamps the Membrane Potential and Supresses Excitability

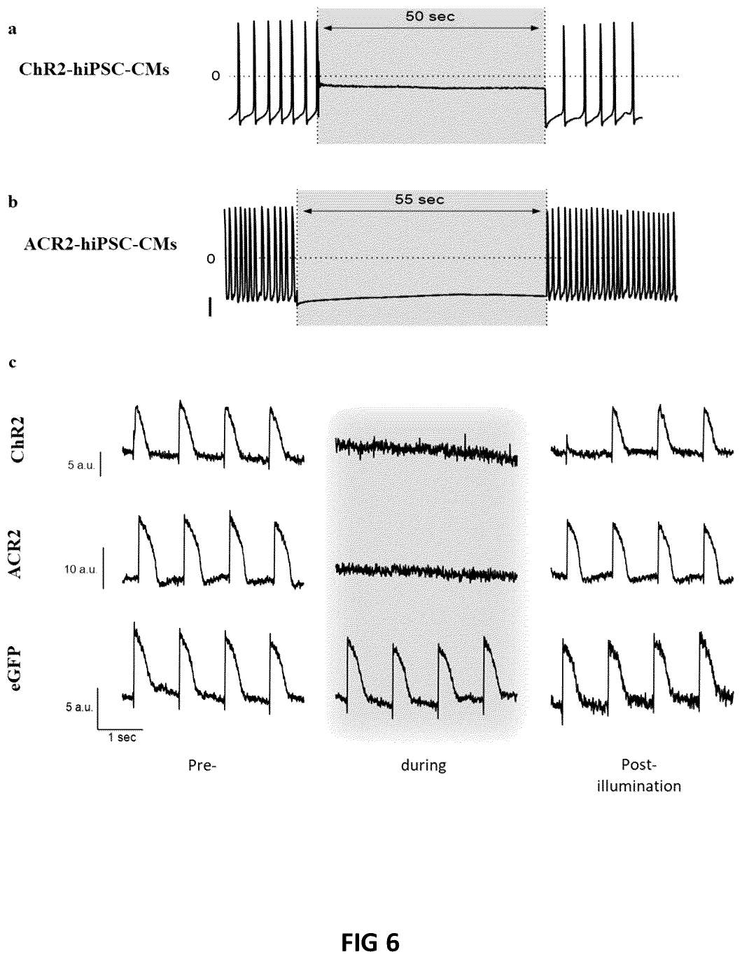

The ability to clamp the membrane potential (Vₘ) by continuous illumination and thereby completely suppress excitability in the hiPSC-CMs was examined. To this end, whole-cell patch-clamp experiments were performed using the current-clamp mode to record APs in hiPSC-CM, which were transduced to express either ChR2 (FIG. 6a) or ACR2 (FIG. 6b). As can be seen in FIG. 6a, prolonged exposure of the ChR2-expressing hiPSC-CMs to 1.3 mW/mm² blue-light illumination clamped the membrane potential to a new and relatively depolarized value and completely supressed the generation of any AP during the illumination period. Upon termination of light stimulation, the cells returned to spontaneously fire APs.

In a similar manner, prolonged exposure of the ACR2-expressing hiPSC-CMs to 1.3 mW/mm² blue-light illumination also clamped Vₘ, but in this case to a very negative (hyperpolarized) potential (FIG. 6b). Interestingly, clamping Vₘ to such a negative potential also completely supressed spontaneous AP generation in the treated cells, with resumption of spontaneous activity following cessation of illumination (FIG. 6b).

Similar light-induced activation studies were next performed in hiPSC-CMs, transduced to express either ChR2, ACR2 or eGFP, which were also loaded with voltage-sensitive dyes to allow monitoring of the optical APs during the experiment (FIG. 6c). The cells were continuously paced by field stimulations at 1 Hz. Importantly, while the ChR2 and ACR2 expressing hiPSC-CMs were successfully paced at this rate at baseline (during darkness), their electrical activity was completely supressed during the prolonged illumination period despite continuous delivery of the electrical stimulus (FIG. 6c, top and middle panels).

Such complete suppression of excitability was noted in 6 out of the 7 ChR2-expressing hiPSC-CMs and in all 19 ACR2-expressing hiPSC-CMs studied. This effect was completely reversible upon cessation of illumination, with resumption of electrical pacing capture and generation of APs (FIG. 6c, top and middle panels). In contrast, in the control eGFP-expressing hiPSC-CMs (n=17), the continuous application of blue-light did not affect the cells' excitability at all, with the electrical pacing stimuli continuing to capture and generate APs in the hiPSC-CMs also throughout the illumination period (FIG. 6c, bottom panel).

### Example 7. Co-Culture Model

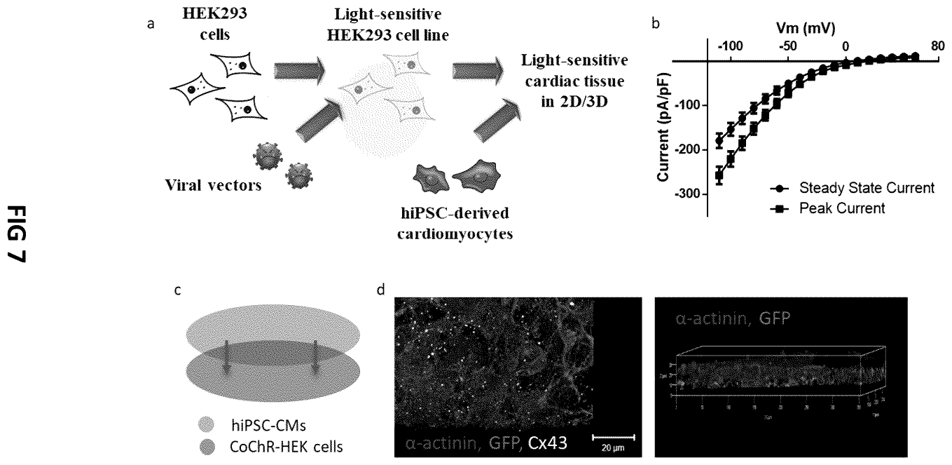

To gain optogenetic control over multicellular tissue models, an opsin-donor engineered cell-line, that can be co-cultured with cardiac myocytes, was established. The engineered cell-line can electrically couple with the adjacent cardiac myocytes and transmit the optic stimuli to them via electrotonic interactions. This concept was previously described, and it suited needs as a robust and effective method to deliver chosen opsins to multilayered tissue models (FIG. 7a). HEK293 cells were transduced with lentivirus carrying a construct of choice: pLV-CAG-Optopatchi or pLV-CAG-CoChR. Both constructs carry ChR2 variants with similar effects, CheRiff or CoChR, respectively. Transduced cells were selected initially using 800 mg/ml G418 for 10 days antibiotic selection and then by fluorescence-activated cell sorting (FACS). The cells were cultured under G418 selection until experiments were conducted. To evaluate the functional photocurrents of the engineered cell-line, whole cell patch-clamp experiments were performed on single CoChR-expressing cells (FIG. 7b, n=5).

Following the satisfactory results from the patch-clamp experiments, a unique two-layered co-culture model of 2-dimensional cardiac tissue (FIG. 7c, 7d) was established. In this model, the engineered cells were previously treated with Mitomycin C to prevent further multiplication. A second layer of hiPSC-CMs was then seeded on top of the first, and cultured to establish the electrotonic interactions with the adjacent HEK293 cells. Immunostaining confirms the co-existence of cardiac myocytes (a-actining positive) and engineered cells (GFP positive), as well as the expression of connexin proteins (Cx43), which form electrotonic connections between cells.

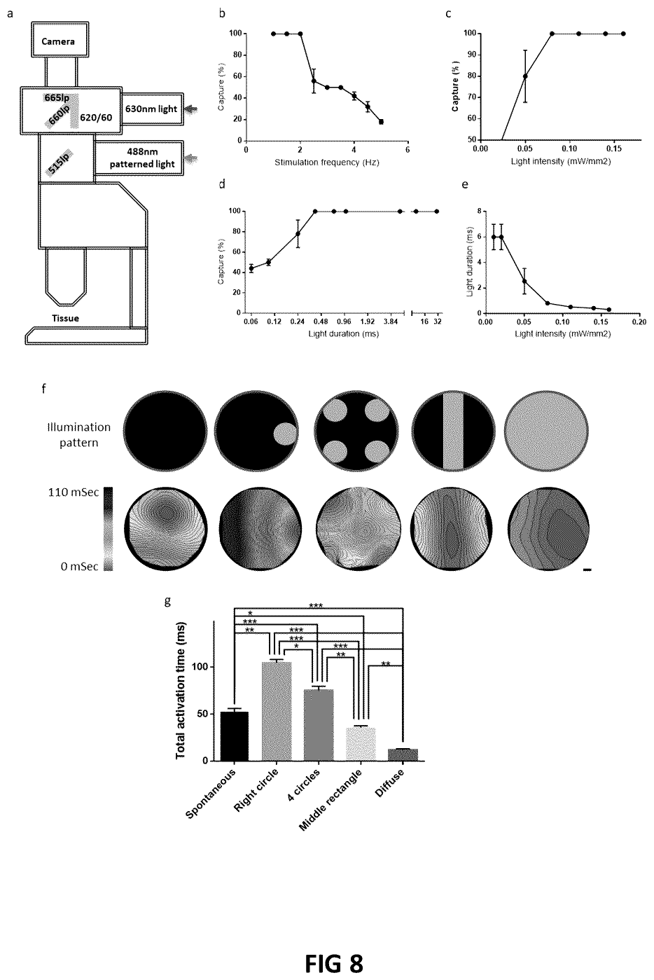

Next, the ability of optical stimulations to trigger activity in the tissue was evaluated. To that end, the tissues with the voltage sensitive dye (VSD) Di-4-ANBDQBS were loaded, and used with previously described EM-CCD-based optical mapping system to monitor electrical activity in the tissues. In addition to the 630 nm light necessary for excitation of the VSD, the system also included a digital micro-mirror device (DMD) to allow spatial patterning of 488 nm light, used for optogenetic stimulation (FIG. 8a).

The initial characterization of the new co-culture model assessed the capture rate of diffuse illumination pulses at variable conditions. 1 ms-long illuminations of 0.16 mW/mm² at 1 Hz was defined as standard, and then changes in frequency (FIG. 8b), stimulation duration (FIG. 8c), and illumination intensity (FIG. 8d) were evaluated. The minimal light duration and intensity for 90% capture rate at 1 Hz was characterized as well (FIG. 8e). Capture rate was calculated for each tissue out of 10 consecutive stimulations, and the mean capture rate ±SEM is presented. n=5.

Further characterization aimed to control geometrical properties of activation and conduction was performed. The response to different illumination patterns that were designed to control the origin of activation and direction of propagation was monitored. The response in the tissue tightly correlated with the applied patterned illumination; the origin of activation took the shape of the illuminated areas, and the propagation extended to the unilluminated areas. This is demonstrated by the activation maps, which display the maximum dF/dt timing in 5 ms isochrones (FIG. 8f). The total activation time (TAT) was calculated as the time from stimulation until excitation is detected throughout the entire tissue area, and as such it reflected the degree of synchronization in the tissue. For example, the TAT was 12.0±1.2 ms when the activation in the tissue was synchronized by diffuse illumination, but was significantly longer (104.6±3.6 ms) when the stimulation pattern was confined to a single circle on the right side of the tissue (FIG. 8g, n=8, P<0.0001).

It was aimed to translate this spatial control over excitation to an ability to model pathological patterns of electrical activation. As demonstrated here below, waves of electrical propagation can be designed to interact with each other, and create conduction blocks and reentrant waves. It was also aimed to synchronize activation of distant areas within the tissue in a similar manner to cardiac resynchronization therapy (CRT). CRT utilizes synchronous electrical stimulation to ameliorate heart failure and mechanical desynchrony in patients, and it was aimed to recapitulate its effects by optogenetics means in-vitro.

### Example 8. Optogenetics-Based APD Modulation at the Tissue Level

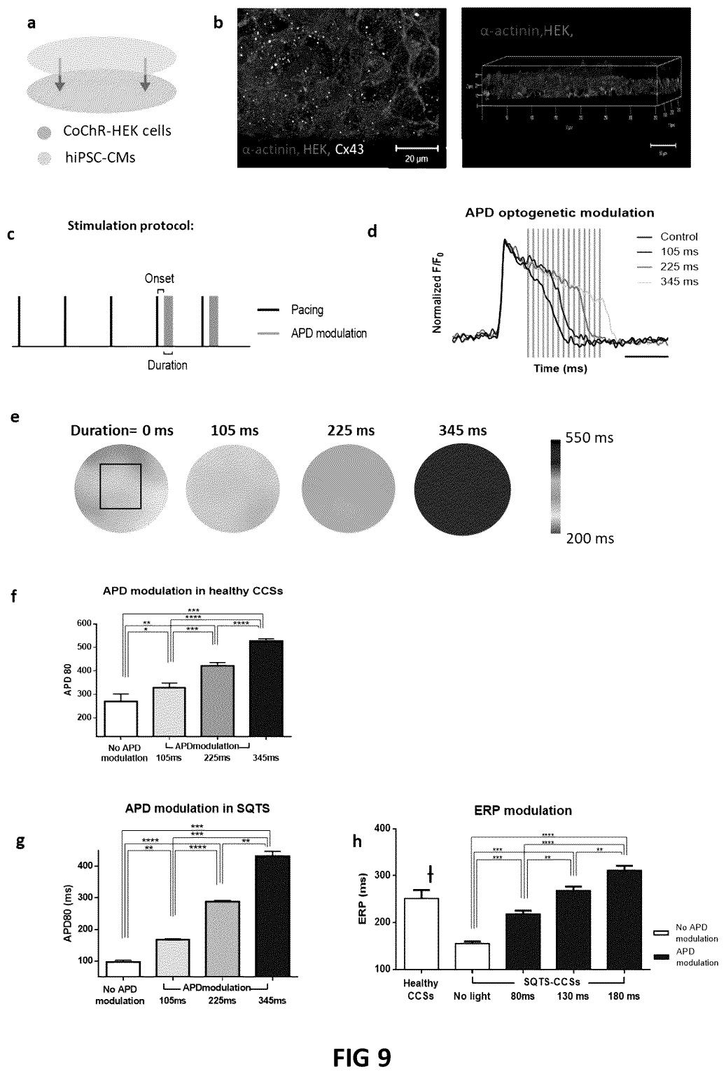

The inventors next aimed to optogenetically control AP properties also at the tissue level. To this end, the inventors utilized a recently-described large-scale (˜1 cm) circular-shaped hiPSC-derived cardiomyocyte cell sheet (hiPSC-CCS) model and co-cultured the layer of cardiomyocytes on top of a monolayer of HEK293 cells, engineered to express the potent chanelorhodopsin variant, CoChR (FIGS. 9a-b). Since HEK293 cells can generate gap-junctions with neighbouring cardiomyocytes, as manifested here by the positive connexin-43 immunostaining (FIG. 9b), CoChR light-activation in the engineered cells is expected to modulate AP properties of the coupled cardiomyocytes through electrotonic interactions.

To monitor the electrical activity of the CoChR-HEK293/hiPSC-CCS co-cultures, they were loaded with voltage-sensitive dyes and evaluated with a high-resolution optical mapping system. To test various optogenetic protocols, the inventors utilized a digital micro-mirror device (DMD), allowing the generation of complex spatiotemporal illumination patterns. The protocol used to test optogenetic-based APD modulation included delivery of four consecutive diffuse optogenetic pacing stimuli [10 ms-long flashes] with the last pacing-stimulus immediately followed (onset-120 ms) by the APD-prolonging optogenetic signal (FIG. 9c). As exemplified in the resulting optical APs traces (FIG. 9d) and APD₈₀ map (FIG. 9e), light-induced CoChR activation significantly prolonged the tissue's APD, with the degree of APD elongation correlating with optogenetic signal duration (FIGS. 9d-f). Consequentially, average APD₈₀ values in the CoChR-hiPSC-CCSs increased significantly from a baseline value of 270±31 ms to 328±20, 422±14, and 527±10 ms following illumination durations of 105, 225 or 345 ms respectively (FIG. 9f).

The inventors next evaluated the ability to optogenetically correct abnormal tissue AP properties by co-culturing CoChR-HEK293 cells with patient-specific SQTS-hiPSC-CCSs. As expected, APD₈₀ values in the SQTS-hiPSC-CCSs were extremely short (98±6 ms, FIG. 9g, n=4) in comparison to healthy-control tissues (270±31 ms, FIG. 9f, n=6). Using the above-mentioned optogenetic APD-modulating protocol (FIG. 9c), the inventors could diffusely prolong and normalize APD₈₀ values in the SQTS-hiPSC-CCSs (FIG. 9g). Thus, application of APD-modulating stimuli (illumination-durations: 105, 225, and 345 ms) significantly prolonged the pathological APD₈₀ values in the SQTS-hiPSC-CCS to 168±3, 288±4, and 431±16 ms respectively (FIG. 9g).

Since a short refractory-period is a key mechanism supporting reentrant arrhythmias in SQTS, the inventors also evaluated the effects of optogenetic APD-modulating protocols on the tissue's effective refractory-period (ERP). The SQTS-hiPSC-CMs exhibited shortened ERP values (170±6 ms, n=5. FIG. 9h) compared to healthy-control tissues (251±18 ms, n=11, FIG. 9h, p<0.01). Using the CoChR-optogenetic APD-modulating signal, the abbreviated ERP in the SQTS-hiPSC-CCSs could be significantly prolonged to 233±7, 283±9 and 326*10 ms using illumination durations of 80, 130, and 180 ms respectively (FIG. 9h, n=5).

### Example 9. Dynamic Optogenetic-Based APD Modulation for Prevention of Reentrant Arrhythmias in the SQTS-hiPSC-CCS Model

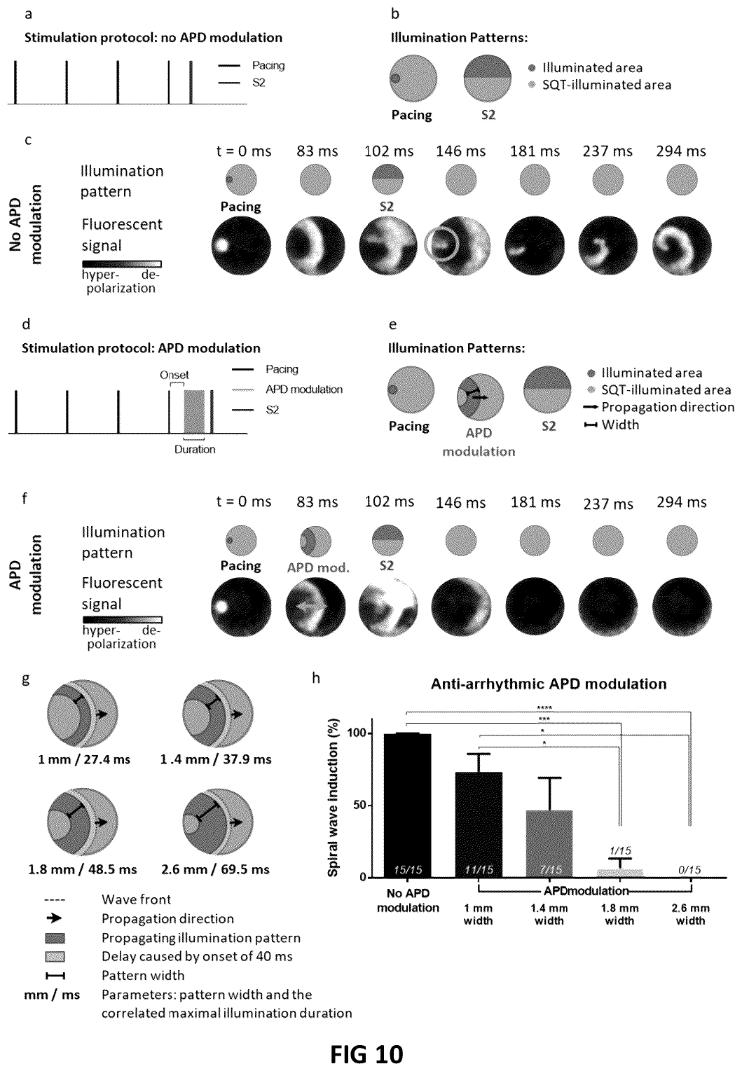

Based on the ability to prolong APD and ERP at the tissue level, the inventors next aimed to develop a dynamic APD-modulating optogenetic strategy that could prevent reentrant arrhythmias. To this end, the inventors initially developed a reproducible arrhythmia model using an optogenetic modification of the well-established electrical cross-field stimulation. This optogenetic protocol, which is schematically outlined in both time and space in FIGS. 10a-b, allowed the generation of reentrant activity in SQTS-hiPSC-CCSs co-cultures in a robust, reproducible, and controlled manner.

An example of how a reentrant activity is induced by the aforementioned approach can be appreciated by the optical mapping results, presented by sequential fluorescent images (FIG. 10c). Note that the SQTS-hiPSC-CCS/CoChR-HEK293 co-culture is initially optogenetically paced using a point-stimulation (S1) originating from the left-side of the culture. When the S1-derived wavefront reaches the center, a broad perpendicular wavefront is initiated from the top of the culture by an S2 optogenetic stimulus. The S2 propagating wave then impinges on the tale of the S1 activation-wave, initiating spiral-wave reentry. Using this approach, the inventors were able to generate sustained reentry repeatedly in all SQTS-hiPSC-CCSs co-cultures (n=15 from 5 independent experiments; FIG. 10c).

The inventors next tested the hypothesis that a dynamic APD-modulating optogenetic-based protocol can be formulated that can prevent reentry in the SQTS-hiPSC-CCS model. To this end, the inventors utilized the DMD apparatus to deliver a dynamic optogenetic stimulation protocol, which was patterned to faithfully follow the S1 propagating wavefront in both time (delivered at each pixel with a 40 ms delay after AP onset) and space; propagating in the same direction and with the same conduction-velocity (CV) as the activation wavefront (FIGS. 10d-g). Application of this dynamic illumination protocol increased both the APD and the resulting tissue wavelength (WL, a product of CV and APD) of the propagating wave, as depicted in the respective fluorescent maps (FIG. 10f, double-headed arrow).

To evaluate whether the aforementioned intervention can prevent arrhythmia-initiation in the SQTS-hiPSC-CCSs, the inventors repeated the same cross-field stimulation protocol but added the dynamic APD-modulating illumination (FIGS. 10d-f). As depicted in the resulting optical-mapping dynamic display and sequential fluorescent images (FIG. 10f), the APD-prolonging protocol was able to prevent initiation of spiral-wave reentry by S2-activation. This anti-arrhythmic effect resulted from the increase in the size of the refractory tissue at the tail of the S1-propagating wave, preventing re-excitation of this proximal area by the S2-derived activation-wave and consequentially its ability to induce reentry.

The inventors next tested the effects of altering the parameters of the dynamic illumination protocol on its anti-arrhythmic capabilities. Specifically, the inventors compared the arrhythmogenic outcome of the cross-field stimulation protocol that was not supplemented by an APD-modulating illumination protocol (control-group) to 4 protocols that also included dynamic APD-prolonging interventions of various illumination-durations (FIG. 10g). Both APD prolongation (in ms) and the associated WL prolongation (in mm) resulting from the different optogenetic interventions are schematically highlighted in FIG. 10g. The results revealed development of spiral-waves following cross-field stimulation in all control (without APD modulation, n=15) experiments. Application of the dynamic APD-modulating protocols significantly reduced the incidence of arrhythmia induction (FIG. 10h). Interestingly, there was a high correlation between the degree of WL increase and the intervention's anti-arrhythmic potential (FIG. 10h). For example, while an illumination protocol designed to increase the WL by 1 mm was associated with a 73% incidence (11/15) of spiral-wave induction, increasing the tissue WL by 2.6 mm completely inhibited (0/15) arrhythmia inducibility (FIG. 10h).

### Example 10. Induction of Spiral Waves in hiPSC-CCSs

The next aim was to characterize the feasibility of optogenetics to control arrhythmias. Specifically, interest was focused in spiral waves as drivers of both atrial and ventricular fibrillation. Given the accumulating data on optogenetic defibrillation in animal tissues, it was sought to expand the understanding in the field to hiPSC-derived cardiac tissues.

First, different arrhythmogenic protocols to induce spiral wave activity were evaluated. In a burst pacing protocol, 5, 10 and 15 repeats of diffuse light pulses (9 ms illumination, cycle length of 24 ms) were applied 120 ms after the pacing stimulation. Only the 15 repeats version of this protocol was able to induce arrhythmia in 1 out of 8 tissues.

In a diffuse S1S2 protocol, specific timing of premature S2 stimulation could exploit intrinsic heterogeneities in the tissue and trigger re-entrant activity. FIG. 9a displays color-coded fluorescent signal, which emphasizes the local differences in membrane potential right before and after S2. Note that such heterogeneity was not present before or after S1. The heterogeneity around S2 gives rise to an unorganized and stable re-entrant activity in the tissue, with multiple cores of spiral waves (t=209 ms and onwards). The efficiency of this protocol depended on the S1S2 interval, or S2 onset. Only S2 onset of 150-200 ms could trigger arrhythmia in the tissues, with a peak efficiency of 55.6% when S2 onset was 175 ms (FIG. 9b, n=9).

To achieve a more robust and uniform provocation of arrhythmia, a cross-field protocol from previous works in the field was adopted. In this protocol, the tissue is paced by a focal illumination pattern (FIG. 9c). This stimulation pattern creates a propagating wave across the tissue, and as a result, a gradient of refractoriness is formed. Then, the premature stimulation S2 occurs, and stimulates half of the tissue in perpendicular to the direction of the propagating wave. If the S2 onset is well timed, as can be seen in FIG. 9d when t=318 ms, the S2 stimulation is able to trigger an origin of activation on the left side of the refractoriness gradient (marked with an arrowhead), but not on its right side. This origin of activation is forced to propagate downwards towards the polarized area (color-coded in blue), but its propagation upwards into the depolarized area (color-coded in green) is blocked by the recent excitation there. Conduction blocks are marked with dashed lines. Ultimately, the propagating wave is guided by the functional conduction blocks to take a route (marked with a grey arrow) around a core point. This protocol imitates the unidirectional block mechanism behind spiral wave initiation, and hence offers very high efficiency in producing much more uniform re-entry patterns than the other protocols. Statistical analysis revealed 100% induction rate of spiral waves when the S2 onset was 250-375 ms (FIG. 9e, n=8).

### Example 11. Spiral Wave Cessation by Optogenetic Illumination

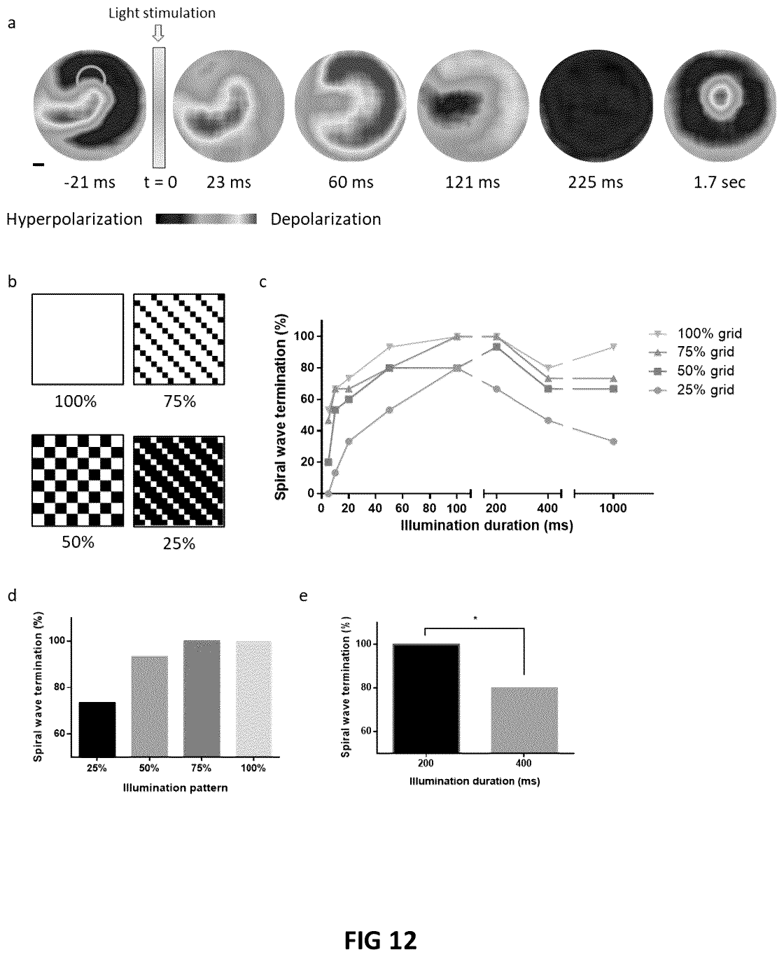

Following establishment of a reproducible optogenetic-based approach to induce reentrant arrhythmias in the hiPSC-CCS, the inventors next aimed to evaluate the ability of optogenetic interventions to terminate such spiral waves. To this end, the inventors applied diffuse continuous illumination to the co-culture, which was able to terminate the propagating spiral wave (FIG. 12a). The mechanism underlying the termination of rotor activity in the culture was related to the illumination-induced depolarization of the tissue that was in a relatively hyperpolarized (excitable) state at the moment of light delivery (FIG. 6a at t=23-60 m). The illumination-related tissue depolarization prevented the propagation of the rotor, eventually leading to spiral-wave termination (FIG. 12a at t=60-225 ms) and to the resumption of spontaneous focal activity (FIG. 6a at t=1.7 sec).

The inventors next aimed to minimize the illumination area required for arrhythmia termination by designing diffuse illumination patterns in which different percentages of the visual field are exposed to light (FIG. 12b). FIG. 12c summarizes the success rate of each illumination pattern in terminating the spiral waves as well as the effect of altering illumination durations. Notice that peak termination rates for each illumination pattern (n=15) were noted when using illumination durations of 100-200 ms durations, reaching as high as 100% termination rates for the 100% and 75% illumination area and 90% rate for 50% illumination area. The effects of altering illumination area can be appreciated in FIG. 12c (for the entire spectrum of illumination durations) and in FIG. 12d (comparing successful termination frequency at 200 ms when the termination rate was maximal). Notice that while 100% illumination area resulted in the highest conversion rate, using 75% or 50% illumination areas were associated with very similar termination rate curves, which did not differ statistically from the 100% illumination area curve. Interestingly, even when using 25% illumination area, although achieving slightly lower termination rates, illumination still resulted in significant termination efficiency. For example, at 200 ms illumination duration (associated with the most efficient termination rate), the arrhythmia termination rate in the 25% illumination area, albeit statistically lower (FIG. 12d, n=15, P<0.0001) than the conversion rates achieved by 100%, 75%, and 50% illumination areas (resulting in 100%, 100% and 95% termination rates respectively), still reached a rather high value of 73%.

The inventors noticed the existence of an optimal “defibrillation time-window”, in which maximal termination rate of spiral waves could be achieved (FIGS. 12c and 12e). Interestingly, not only that short illumination durations (≤550 ms) were less effective in terminating reentry, but also very prolonged illuminations durations (≥400 ms) revealed reduced efficacy. This can be appreciated, for example, in FIG. 6e showing the significantly higher arrhythmia termination rate at 200 ms illumination duration (100% success rate) as compared to 400 ms illumination duration (80% success rate) when using a100% illumination grid (FIG. 12e, n=15, P<0.05).

### Example 12. Spiral Wave Cessation by Optogenetic Illumination in a 3D Engineered Heart Tissue Model

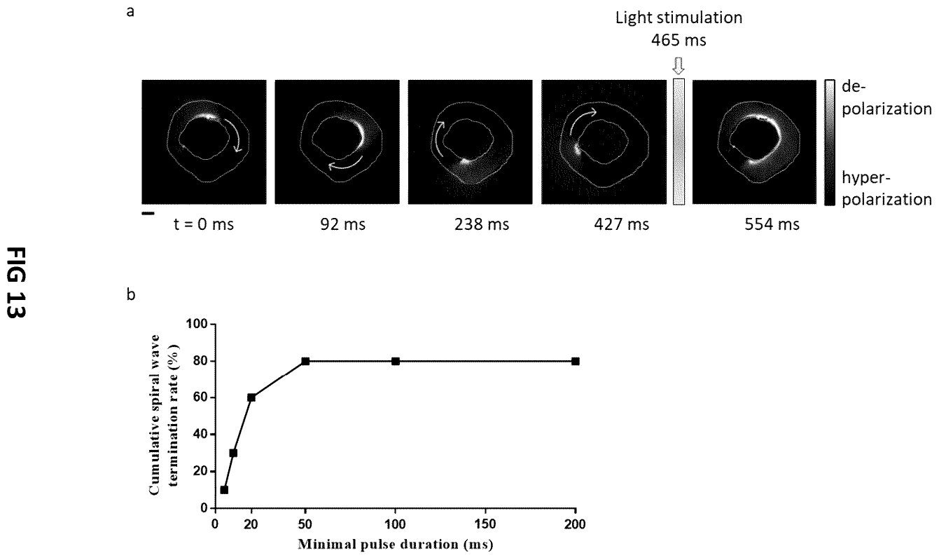

The inventors also evaluated the ability to terminate reentrant activity also in the three-dimensional hiPSC-CMs/CoChR-HEK393 EHT model describe above. Following induction of reentrant activity in the circular EHT model using burst electrical stimulation, the inventors delivered a diffuse optogenetic stimulation to the tissue (FIG. 13a). This resulted in depolarization of the non-depolarized tissue that was not activated at the time of illumination (FIG. 13a 465-554 ms). The resulting depolarization prevented the propagation of the reentrant wavefront, leading to termination of the arrhythmia. The inventors next evaluated the effect of prolonging the optogenetic stimulation duration on the cumulative reentry termination rate and noted a plateau at 50 ms in which 80% of the arrhythmic events were terminated (FIG. 13b, n=10).

### Example 13. APD Homogenization by Optogenetic Illumination in hiPSC-CCSs

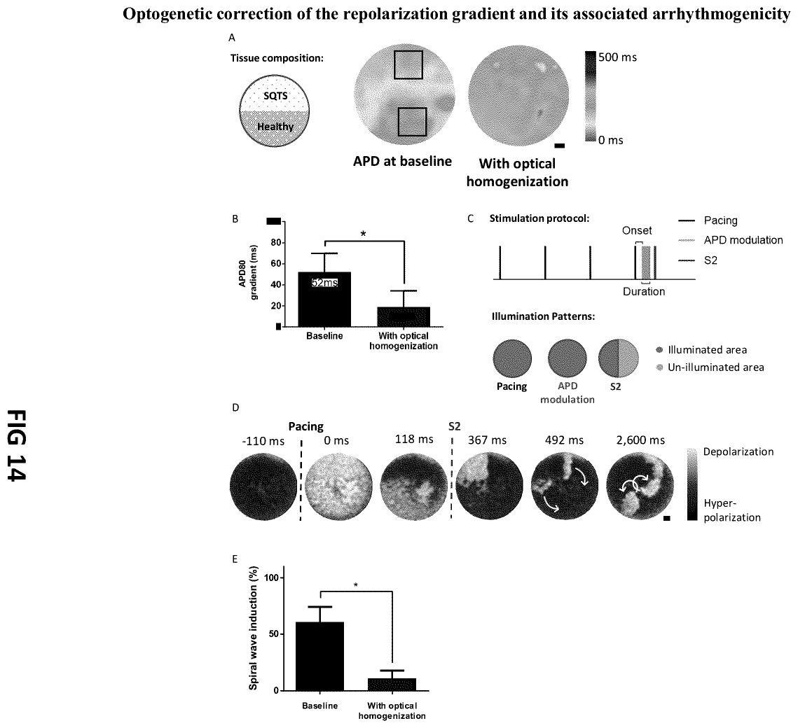

Optogenetics was used to reduce APD gradients in the tissue and tested whether such intervention can reduce the arrhythmogenicity associated with such gradients. For those experiments, they established a tissue model that is comprised of two hiPSC-CM lines: healthy and SQTS. The two types of cardiomyocytes had inherent differences in their APD, and when the inventors cultured them side by side as a tissue they created a localized APD or repolarization gradient (FIG. 14A, B). This situation is known to be associated with an increased arrhythmogenic risk as was also shown in this model. Then, by applying a diffuse 105 ms-long illumination with an onset of 40 ms, the APD gradient in the tissue could be significantly reduced from 52.2±8.8 to 18.9±7.7 ms (FIG. 14B, p<0.01, n=4).

To test the functional implication of the innate APD gradient of the tissues and the optical ability to homogenize that gradient, an S1-S2 protocol with a premature stimulation S2 that covered half of the tissue and was aligned with the APD gradient (FIG. 14C). With carefully selected S2 onsets of 160, 180 and 200 ms, the S2 stimulation could trigger an AP only in the tissue area with the shorter APD and therefore with shorter refractory period. (FIG. 14D, t=118, 367 ms). The premature AP could then propagate to the excitable tissue that recovered from the S1 stimulation (FIG. 4D, t=492 ms, white arrows represent the direction of propagation). The propagating waves then took the form of sustained spiral waves (FIG. 14D, t=2.6 s) that lasted more than 10 seconds and were terminated only after intervention.

In the presence of the innate APD gradient, the spiral wave induction rate from a set of experiments with S2 onsets of 160, 180 and 200 ms was 61.0±13.4%. Nevertheless, when optogenetic homogenization of APD was applied, the same S2 stimulations resulted in a significantly lower spiral wave induction rate of 11.0±7.0% (FIG. 14E, p<0.05, n=6).

A number of implementations have been described. Nevertheless, it will be understood that various modifications may be made without departing from the spirit and scope of the disclosure. For example, various forms of the materials shown above may be used, with steps re-ordered, added, or removed. Accordingly, other implementations are within the scope of the following claims.

The examples presented herein are intended primarily for purposes of illustration of the invention for those skilled in the art, and to illustrate potential and specific implementations of the present disclosure. No particular aspect or aspects of the examples are necessarily intended to limit the scope of the present invention.

The figures and descriptions of the present invention have been simplified to illustrate elements that are relevant for a clear understanding while eliminating, for purpose of clarity, other elements. Those of ordinary skill in the art may recognize, however, that these sorts of focused discussions would not facilitate a better understanding of the present disclosure, and therefore, a more detailed description of such elements is not provided herein.

Unless otherwise indicated, all numbers expressing lengths, widths, depths, or other dimensions and so forth used in the specification and claims are to be understood in all instances as indicating both the exact values as shown and as being modified by the term “about.” As used herein, the term “about” refers to a ±10% variation from the nominal value. Accordingly, unless indicated to the contrary, the numerical parameters set forth in the specification and attached claims are approximations that may vary depending upon the desired properties sought to be obtained.

A number of embodiments of the invention have been described. Nevertheless, it is to be understood that the foregoing description is intended to illustrate and not to limit the scope of the invention, which is defined by the scope of the following claims. Accordingly, other embodiments are also within the scope of the following claims. For example, various modifications may be made without departing from the scope of the invention. Additionally, some of the steps described above may be order independent, and thus can be performed in an order different from that described.

1. A method for modulating action potential (AP) properties of cardiac tissue cells of a patient in need thereof, the method comprising the steps of:
(a) providing cardiac tissue cells in which the membrane potential is susceptible to light-dependent modulation, and
(b) illuminating the cardiac tissue cells during the action potential.

2. The method of claim 1, wherein the method comprises modulating the AP duration (APD) of the cardiac tissue cells.

3. The method of claim 2, wherein the method comprises elongating the APD of the cardiac tissue cells.

4-41. (canceled)

42. The method of claim 3, wherein the method comprises improving the contraction of the cardiac tissue cells or silencing the electrical activity of the cardiac tissue cells.

43. The method of claim 3, wherein the method comprises illuminating the cardiac tissue cells for about 10 to about 2000 milli-seconds, or illuminating at least 50% of the cardiac tissue cells.

44. The method of claim 2, wherein the method comprises depolarizing the cardiac tissue cells, or illuminating the cardiac tissue cells in phase 2 or phase 3 of the AP.

45. The method of claim 2, wherein the method comprises shortening the APD of the cardiac tissue cells.

46. The method of claim 45, wherein the method comprises decreasing the contraction of the cardiac tissue cells or silencing the electrical activity of the cardiac tissue cells.

47. The method of claim 46, wherein the method comprises illuminating the cardiac tissue cells for about 10 to about 2000 milli-seconds, or illuminating at least 50% of the cardiac tissue cells.

48. The method of claim 47, wherein the method comprises hyperpolarizing the cardiac tissue cells.

49. The method of claim 48, wherein the method comprises illuminating the cardiac tissue cells in phase 0, phase 1, phase 2, or phase 3 of the AP.

50. The method of claim 1, wherein the method comprises changing the slope of repolarization of the AP or extending the plateau phase of the AP.

51. The method of claim 1 for preventing or treating Short QT Syndrome (SQTS), Long QT Syndrome (LQTS), early-after-depolarizations (EADs) or triggered beats.

52. The method of claim 1 for preventing or treating cardiac arrhythmia.

53. The method of claim 52, wherein the arrhythmia is polymorphic ventricular tachycardia, Torsade-de-Pointe (TdP), or reentrant arrhythmia.

54. The method of claim 1, wherein the patient is afflicted with a Short QT Syndrom (SQTS), Long QT Syndrom (LQTS), cardiac tissue action potential duration (APD) or repolarization heterogeneity.

55. The method of claim 1, wherein making the membrane potential of the cardiac tissue cells susceptible to light-dependent modulation comprises (a) genetically transforming the cardiac tissue cells with a light-sensitive ion channel, a light-sensitive ion pump, or a light-sensitive signaling receptor, or (b) contacting the cardiac tissue cells with genetically-transformed cells comprising a light-sensitive ion channel, a light-sensitive ion pump, or a light-sensitive signaling receptor.

56. The method of claim 55, comprising:
(a) expressing a light-sensitive ion channel, a light-sensitive ion pump, or a light-sensitive signaling receptor in the cardiac tissue cells;
(b) contacting the cardiac tissue cells with an oligonucleotide construct encoding a light-sensitive ion channel, a light sensitive ion pump, or a light-sensitive signaling receptor;
(c) administering genetically-transformed cells to the patients' heart, wherein the genetically-transformed cells comprise a light-sensitive ion channel, a light-sensitive ion pump, or a light-sensitive signaling receptor; or
(d) administering genetically-transformed cells to the patients' heart, wherein the genetically-transformed cells comprise an oligonucleotide construct encoding a light-sensitive ion channel, a light-sensitive ion pump, or a light-sensitive signaling receptor.

57. The method of claim 56 wherein the light-sensitive ion channel, light-sensitive ion pump, or light-sensitive signaling receptor is selected from the group consisting of ChR2, ChR2/H134, ChETA, ChR/T159C, SFO/SSFO, ReaChR, VChR1, Chronos, Chrimson, ChrimsonR, PsChR2, CoChR, CsChR, CheRiff, C1C2, ChIEF, ChEF, ChD, C1V1, iChloC, SwiChRca, GtACR, PsChR1, Phobos, Aurora, Jaws, Halo/NpHR, eNpHR 3.0, Arch, eArch 3.0, ArchT, ArchT 3.0, Mac, and eMac 3.0.
